(19) United States
(12) Patent Application Publication
(10) Pub. No.: US 2025/0255894 A1
(43) Pub. Date: Aug. 14, 2025

(54) INHIBITORS OF EIF4A

(71) Applicant: ACEAE NUTRA LIMITED, Hadleigh, Ipswich (GB)

(72) Inventors: Andrew Bottley, Hadleigh, Ipswich (GB); Natalie Chapman, Hadleigh, Ipswich (GB)

(21) Appl. No.: 18/845,260

(22) PCT Filed: Mar. 8, 2023
(86) PCT No.: PCT/GB2023/050547

§ 371 Date: Sep. 9, 2024

(30) Foreign Application Priority Data
Mar. 8, 2022 (GB) 2203181.9

(51) Int. Cl. A61K 31/7032; A61K 45/06; A61P 31/14; A61P 31/16; C07H 15/04

(52) U.S. Cl. CPC A61K 31/7032; A61K 45/06; A61P 31/14; A61P 31/16; C07H 15/04

(57) ABSTRACT

A compound of Formula (I), or a composition thereof. The compound or composition thereof is for use as a medicament, for example for use in a method of treatment or prevention of a viral infection or a CNS-related condition, or used to increase the yield of an animal or herd of animals, or to improve the appearance of the animal or herd thereof.

### FIELD

The present invention relates to inhibitors of eukaryotic initiation factor 4A (eIF4A). In particular, the present invention relates to eIF4A inhibitors, and compositions and formulations thereof, for use as a medicament, for example as an antiviral agent and/or as a treatment or prevention of a CNS-related condition. The use of such compounds, and compositions thereof, in agriculture is envisaged.

### BACKGROUND

The transmission of infectious diseases, such as viral infections, through a herd of animals can significantly reduce yield of product obtained from the heard. For example, bovine respiratory disease (BVD) has been cited as the principal source of economic loss for the North American beef industry, and this also is a significant health problem in the dairy industry. The pathogenesis of BVD typically involves some combination of predisposing stress which compromises respiratory defence mechanisms and coincidental primary infection with one or more respiratory viruses.

The treatment of a variety of viral infections (e.g. influenza) has been achieved with inhibitors of eukaryotic initiation factor 4A (eIF4A). eIF4A is a DEAD-box helicase that is part of the cellular eIF4F translation initiation complex. The main functions of eIF4A are to remove secondary complex structures within the 5′-untranslated region and to displace proteins attached to mRNA.

Owing to the critical role of eIF4A in protein synthesis, eIF4A can be inhibited to treat and/or prevent a wide variety of diseases. eIF4A has been well documented in regulating the synthesis of stress response proteins in plants and equivalent roles reported in human and animal studies. There is now compelling evidence that aberrant control of protein synthesis is linked to the progression of a range of other conditions and illnesses.

eIF4A inhibitors have been developed to treat other diseases and conditions, including anti-inflammatories, treatments for cancer, treatments for conditions of the central nervous system and longevity.

Thus, eIF4A inhibitors are sought-after for the treatment of viral infections and other diseases that can be controlled by the inhibition of eIF4A.

Mounting an immune response is a protein intensive process and once initiated requires well co-ordinated and tightly regulated protein synthesis. Disrupting the initiation of the RNA translation step has been shown to be anti-immunogenic and the role of UTR scanning performed by eIF4A can be rate limiting in the synthesis of pro-inflammatory cytokines such as Interleukin 6 (IL-6) (doi: 10.1158/0008-5472.CAN-14-2789). Proteins such as IL-6 have been implicated in the pathogenesis of many different diseases and conditions e.g. Atopic dermatitis, Autism Spectrum Disorder (i.e. autistic spectrum disorders), Psoriasis and Cachexia; experiments have shown that inhibition of eIF4A co-ordinately suppresses pathways associated with disease e.g. Cachexia-eIF4A inhibition prevents onset of cytokine-induced muscle wasting by blocking the STAT3 and iNOS pathways in-vitro (Scientific Reports volume 8, Article number: 8414, 2018), and these results have been replicated in appropriate in-vivo models for this condition (doi: 10.1038/ncomms1899)

It is therefore unsurprising that eIF4A has been proposed as a target for treating or ameliorating both acute and chronic inflammatory conditions; pathways known to be regulated by inhibitors of eIF4A include e.g. (but not limited to):
(a) small molecule inhibitor of eIF4A 15d-PGJ2 ‘resets’ aberrant immune activity (e.g. IL-4) and is a naturally occurring anti-inflammatory, produced by a range of cell types and found in human body fluids. 15d-PGJ2 (30-100 μg/kg, s.c.) decreases house dust mite extract (HDM)-induced cytokine and chemokine production in lung tissue samples.
(b) Selective increase of global protein synthesis in endogenous inhibitor of eIF4A PDCD4 knockdown−/− cells correlates with relative increase in levels of induced pro-inflammatory Interleukins IL-4 and IL-10.
(c) Hippuristanol, a compound that inhibits eIF4A inhibits the iNOS/NO pathway and inflammatory cytokine-induced muscle wasting. Further, treatment with hippuristanol supresses the activation of the STAT3 pathway and expression of STAT3-gene targets such as IL-6 see Scientific Reports volume 8, Article number: 8414 (2018).

Furthermore, longevity can be provided by inhibiting eIF4A. Protein synthesis is one of the most important processes in the cell. Correct maintenance of the proteome in any organism requires an accurate regulation of synthesis and degradation of each and every protein; conversely aging often shifts this balance, negatively impacting on longevity (Cold Spring Harb Perspect Biol. 2011; 3:3, doi: 10.1101/cshperspect.a004440). A study conducted by Curran and Ruvkun (doi: 10.1371/journal.pgen.0030056) screened by siRNA knockdown 2,700 genes essential for Caenorhabditis elegans development. They identified 64 genes that extend lifespan when inactivated post-developmentally. Of those genes identified as positively impacting on longevity, eIF4A is found twice in the list of top 25 genes showing most positive impact on lifespan (P value<0.0001) (two different siRNA sequences targeting the same protein). Inhibition of eIF4A therefore correlates with extended longevity in living organisms.

Experimental evidence indicates that inhibitors of translation or compounds which act to modify or alter protein synthesis present an attractive opportunity as broad acting antivirals. In particular, there is evidence to show inhibitors of eIF4A provide pan-virus antiviral activity (including RNA+, RNA− and DNA viruses) (Microorganisms., 2021 March; 9(3): 540). Inhibition of eIF4A using compounds such as silvestrol, has also been shown effective against Ebola virus, MERS-CoV, HCoV-229E, Poliovirus type 1, Chikungunya virus, Hepatitis E, Zika virus, Influenza A virus, Lassa virus and Crimean-Congo haemorrhagic fever virus, Hepatitis C virus, Enterovirus (Eur J Med Chem., 2020 Oct. 1; 203: 112653).

For example, hippuristanol has been shown to effectively disrupt the control of HIV virus translation. Indications from studies using inhibitors of protein synthesis such as hippuristanol suggest that this is a relatively non-toxic treatment option.

With regard to the human immunodeficiency virus (HIV), the relative expression of the two isoforms p55 and p40 of HIV-1 Gag proteins is highly dependent on the correct functioning of the translation initiation complex. The highly structured 5′-UTR of the viral p55 gene has been shown to tightly control of expression through a requirement of the eIF4F complex, especially the RNA helicase eIF4A (de Breyne et al, 2012. FEBS J. 279, 3098-3111). Additional research performed using the known inhibitor of eIF4A hippuristanol has evaluated the requirement for eIF4A in the correct translation of HIV proteins. Increasing amounts of hippuristanol inhibits the translation of the three Gag isoforms in a similar dose-response manner thus confirming a functional requirement for eIF4A in the HIV life cycle (Locker et al, 2010. Nucleic Acids Res. 39, 2367-2377). Recent work by Plank et al., (2014. Vol. 2, Iss. 1) confirmed that hippuristanol treatment of HeLa cells transfected with HIV-1 leader constructs inhibited IRES activity, with IC50 values in a drug-gable range (163 to 296 nM). Taken together, these results confirm that eIF4A is important in the HIV life cycle and that eIF4A presents an attractive new therapeutic target for this virus.

Herpes simplex virus HSV-1 has been shown to stimulate eIF4E phosphorylation and eIF4F complex formation in resting primary human cells. It is also known that the VHS protein (virion host shut-off), an HSV viral endonuclease, selectively associates with eIF4A and eIF4H during the viral life cycle. In addition to degrading host mRNAs, VHS is thought to play a role in regulating the temporal pattern of viral mRNA expression, through enhancement of viral RNA translation. VHS associates with eIF4A/eIF4H and, despite its endonuclease activity, this association with eIF4A has been shown to enhance translation from viral IRES (internal ribosome entry site) elements and sequences within HSV-1 5′-UTRs (Saffran et al, 2010. J. Virol. 84, 6041-6049; Reviewed by Walsh, D. (2010). Biochem. Soc. Trans. 38, 1511-1516.).

Inhibitors of eIF4A have been shown to have value in the prevention of influenza viral replication (e.g. WO 2013/152299). Recent research has demonstrated the functional impairment of eIF4A correlates with inhibition of influenza virus mRNA translation and protein synthesis, and that this helicase is essential for viral translation (data obtained from both in in vivo and in vitro analysis) (Yángüez et al, 2011. Virology. 413, 93-102). Viral mRNAs have been shown not to contain cis-acting signals that may mediate eIF4A independent translation and it is also known that trans-acting viral proteins cannot replace the function of mammalian eIF4A. Therefore, inhibition of eIF4A is an attractive target to prevent the propagation and replication of the influenza virus in infected cells.

Viruses such as influenza result in substantial agronomic loss when they infect livestock. A treatment to reduce the severity of infection can mitigate loss and improve yield. For example, an outbreak of avian flu in Manipur, India, in 2007 resulted in a 14 percent state-wide reduction of the total value of livestock outputs (Agricultural Economics Research Review, Vol. 21 January-June 2008 pp 37-47). The prevention and/or treatment, even if resulting in a reduction of symptoms of the viral infection rather than a cure, of such viruses is therefore particularly valuable.

Coronaviruses (e.g. human coronaviruses, including SARS-CoV, MERS-CoV and SARS-CoV-2) are recognized to cause up to a third of common colds and are also the cause of severe viral infections such as SARS. Coronavirus replication involves the generation of mRNAs with capped 5′UTRs. Coronavirus 5′UTRs, such as those identified from SARS isolates, are relatively well conserved and the full sequence forms a complex secondary structure containing four stem-loop domains. As 5′UTR secondary structure directly correlates with the requirement for eIF4A, it is not surprising that eIF4A is considered a therapeutic target for coronavirus infection.

The translation of most of the coronaviral mRNAs is thought to be cap dependent and requires a functional translation initiation complex eIF4F (Cencic et al, 2011. J Virol. 85, 6381-6389). Inhibition of translation with the eIF4A inhibitors hippuristanol or silvestrol caused a 10- to 100-fold reduction in infectious coronavirus virus titres released from infected cells (Cencic et al, 2011. J Virol. 85, 6381-6389). This virus has been proven to be dependent on eIF4A and a significant reduction in viral progeny has been observed upon the inhibition of eIF4A (Cencic et al, 2011. J Virol. 85, 6381-6389).

Rhinoviruses are the most common viral infective agents in humans and are the major cause of the common cold. Internal ribosomal entry site elements of poliovirus (PV), human rhinovirus (HRV) and encephalomyocarditis virus (EMCV) foot-and-mouth disease virus (FMDV) groups are all inhibited by disruptive mutations to the eIF4A protein (Svitkin et al, 2001. RNA. 7, 382-394). These viruses are therefore dependent on eIF4A activity.

Human cytomegalovirus (HCMV) is a herpes virus that can have serious and life-threatening consequences for immunocompromised patients. As HCMV infection progresses, the abundance of core eIF4F components (eIF4A is part of the eIF4F complex) greatly increases (Walsh et al, 2005. J. Virol. 79, 8057-8064). In addition, HCMV UL69, homologous with the HSV-1 ICP27 protein, associates with eIF4A (Aoyagi et al, 2010. Proc. Natl. Acad. Sci. U.S.A. 107, 2640-2645). Pateamine A, a known inhibitor of eIF4A, inhibits the replication of HCMV (WO 2013/152299). Disrupting eIF4A activity presents a therapeutic target as an antiviral for HCMV.

There is also good evidence that the initiation of translation of norovirus proteins is dependent on the interaction of the VPg with the translation initiation complex (Daughenbaugh et al, 2003. EMBO J. 11, 2852-2859; Daughenbaughet et al, 2006. Virol J. 23, 3-33). Panteamine A, a proven inhibitor of eIF4A, has the potential to interfere with VPg/eIF4F complex, since it disrupts the helicase/NTPase activity of eIF4A, dysregulating its function within the eIF4F complex (Bordeleau et al, 2006. Chem Biol. 13, 1287-1295). Virologists suggest that inhibitors of eIF4A could therefore be exploited as antivirals for norovirus due to this dependency (See Rocha-Pereira and Nascimento, 2012 Targeting Norovirus: Strategies for the Discovery of New Antiviral Drugs, Antiviral Drugs—Aspects of Clinical Use and Recent Advances, Dr. Patrick Arbuthnot (Ed.), ISBN: 978-953-51-0256-4, InTech).

Inhibition of eIF4A is also expected to prevent and/or treat such viral infections in aquaculture. For example, common viruses and by association viruses in the same families that infect fin-fish and shell fish include Viral haemorrhagic septicaemia virus (VHSV), Infectious haematopoietic necrosis virus (IHNV), Infectious salmon anaemia virus (of Orthomyxoviridae), Piscine orthoreovirus (Orthoreovirus, dsRNA), Tilapia lake virus (RNA−). Also Covert mortality nodavirus (alphanodavirus, RNA+), Shrimp hemocyte iridescent virus (Iridoviridae, dsDNA), and Abalone herpesvirus (Herpesvirales, dsDNA).

Chronic CNS-related conditions such as muscle wasting, autism spectrum disorder, Alzheimer's disease, Huntingdon's disease, frontotemporal dementia, motor neurone disease and Parkinson's disease all share similar patterns of deregulation of protein synthesis and a number of research studies conclude that pharmacological agents targeting the protein synthesis machinery are one potential route to treatment for such conditions.

Recent high impact research into the cause of autism spectrum disorder (ASD) has identified that dysregulation of protein synthesis in neuronal cells at the point of translation initiation is a primary driver of ASD symptoms (Gkogkas et al, 2013 Nature, 2013, 493:371-377; Santini et al, Nature, 2013, 493:411-415).

Gkogkas et al, 2013. Nature, 493, 371-377 demonstrated a direct link between ASD and the relative translation of two neuroligins; these are proteins which mediate new connections between neuronal cells and regulate the composition of neurotransmitter receptors. This new research identifies that the ratio of the synthesis of these two proteins is selectively determined by the activity of the translation initiation complex and that dysregulation of synthesis drives or promotes the symptoms of ASD. Importantly it is the relative synthesis of neuroligin 1 (NLGN1) protein that is incorrectly regulated; therefore, selective control of NLGN1 has been demonstrated to be a viable treatment option for ASD.

Gkogkas et al 2013. Nature, 493, 371-377 describes model therapeutic intervention to regulate NLGN1 is mediated via inhibition of eIF4E, a key protein in the translation initiation complex. However, eIF4A represents an additional and more selective new target for the control of NLGN1 synthesis; a target to elevate the symptoms of ASD. The eIF4A helicase functions to unwind long, complex and structured 5′UTRs; this is required before protein synthesis can begin. Inhibiting eIF4A selectively reduces the synthesis of proteins with greater 5′UTR secondary structure or longer length, while not inhibiting those with short 5′UTRs or unstructured UTRs. Treating cells with the coral-derived inhibitor of eIF4A, hippuristanol, results selective inhibition determined by features present within the 5′UTR (Bottley et al, 2010 PLOS One, 5(9): e13030).

The disruption of one or more steps in the control of protein synthesis has been associated with alterations in the cell cycle and/or regulation of cell growth. Evidence supports the concept that some translation factors are proto-oncogenes and proteins involved in translation pathways can act as key regulators of malignant progression (Hershey et al, 2000 Translational Control and Cancer, Cold Spring Harbor Laboratory Press, Cold Spring Harbor). Cancer cells generally show higher rates of protein synthesis compared to normal cells. Accordingly, deregulation of protein synthesis is emerging as a major contributor to cancer progression. Over-expression of certain translation factors can lead to malignant transformation and many of the components of the translation pathways are over-expressed in cancer. A number of clinically relevant in-vivo experiments have demonstrated that inhibition of translation may be relevant for the treatment of a range of cancer types, e.g. adult T-cell leukaemia, lung, breast and cervical cancer. The requirement for elevated levels of protein synthesis is a common feature of cancer cell growth; therefore, it is highly likely that a wider broad spectrum of cancer types will also be amenable to treatment with this class of inhibitor.

Furthermore, inhibitors of translation have shown remarkable promise for use as an adjuvant therapy in combination with chemotherapeutics such as Doxorubicin™. Rapidly proliferating tumour types such as MCF-7 breast cancer cells require relatively more protein synthesis than slower growing cancer cells such as A549 lung carcinoma cells. These slow-growing cancer cell types have relatively higher patient mortality rates five years after diagnosis due to chemoresistance to common in clinic chemotherapeutics agents such as Cisplatin™. Research has shown that cell types such as A549 lung carcinoma or SKOV3 ovarian cancer cells derive resistance to platinum-based therapies through the aberrant translation of specific proteins such as LARP1. Experimental evidence also suggests that endogenous inhibitors of protein synthesis such as programme cell death 4 (PDCD4) modulate sensitivity to Cisplatin™ and that the levels of these endogenous inhibitors significantly correlate with disease-free survival of ovarian cancer patients.

Therapeutic modulation of protein translation by inhibition of eIF4A is also a proven target for the treatment for a broad range of cancer types. Regulation of protein synthesis at the level of translation initiation (eIF4F complex containing eIF4A) is particularly important in cancer cell growth because they are metabolically highly active. This rapid growth places a heavy demand on the protein synthesis machinery. Additionally, cancer cells often produce proteins that provide resistance to commonly used chemotherapeutic drugs and this resistance is determined by selective translation of key proteins i.e. dependent on eIF4A. De novo or acquired resistance to platinum chemotherapy is the leading cause of death in some cancers, e.g. ovarian. Research identifies that this chemo-resistance is due to the aberrant translation of key proteins (Boussemart et al 2014, Nature, ahead of print doi:10.1038/nature13572; Wolf et al. 2014 Nature, ahead of print doi:10.1038/nature13485; also see reviews by Blagden and Willis, 2011 Nature Oncology Reviews, 8:280-291; Bitterman and Polunovsky 2012, Molecular Cancer Theraputics, 11: 1051-1061).

The therapeutic modulation of mRNA translation by the inhibition of eIF4A is therefore an excellent and well-established intervention point for the treatment of a range of different cancer types, enabling a selective treatment targeted to the biology of the cancer cell. Initiation of translation is a point of convergence for multiple aberrant signalling cascades and represents a logical approach for targeting chemotherapy-resistant cancer cells (cancer types include but are not limited to ovarian, lung, breast, leukaemia, pancreatic, kidney).

Although the need for chemical modifiers of translation has been well established, most current small molecule inhibitors, such as hippuristanol, are sourced from rare marine corals or sponges and prove difficult to synthesise in any meaningful quantity. Such molecules have however been successfully used to provide in-vivo evidence that this class of inhibitor is a likely successful strategy option for use in the clinic, however these molecules are source limited and as such not an available option for clinical use.

An aim of the present invention is to provide novel inhibitors of eIF4A, which could be used as medicaments, in particular as antiviral agents and/or as a treatment for a CNS-related condition.

The invention may provide, in part, compounds for use as antiviral agents and/or as a treatment for a CNS-related condition, and pharmaceutical and nutraceutical compositions including the compounds.

The compounds may be for use in treating diseases and disorders which are linked to aberrant control of protein synthesis. The compounds may find applications as antiproliferative, chemotherapeutic, cell sensitising or adjuvant agents, for example for use in treating cancer.

eIF4A inhibitors that can be obtained from a readily available source would be particularly beneficial.

### SUMMARY

According to a first aspect the present invention provides a compound of Formula (I) for use as a medicament, wherein Formula (I) is:

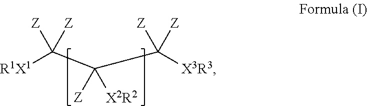

including tautomeric or stereochemically isomeric forms thereof, wherein:
R¹ is selected from a carbohydrate group or a derivative thereof, a C1-C30 alkyl group or a derivative thereof, a C2-C30 alkenyl group or a derivative thereof, and a C2-C30 alkynyl group or a derivative thereof;
a hydrocarbon group is selected from a C1-C30 alkyl group or a derivative thereof, a C2-C30 alkenyl group or a derivative thereof, and a C2-C30 alkynyl group or a derivative thereof;
n is 0 or 1;
where n is 0, R³ represents the hydrocarbon group;
where n is 1, either: a) R² represents H and R³ represents the hydrocarbon group, or b) R² represents the hydrocarbon group and R³ represents H;
X¹ is a linking group;
X² is a linking group;
X³ is a linking group;
each Z group independently represents Y, R^{Z} or H;
each Y group independently represents a group selected from the list consisting of: cyano, halogen, N₃, —C(O)R^{Z}, —C(O)OR^{Z}, —OC(O)R^{Z}, —C(O)NHR^{Z}, —NHC(O)R^{Z}, —NHC(O)NHR^{Z}, —NHC(O)OR^{Z}, —OC(O)NHR^{Z}, —OP(O)₂OR^{Z}, —S(O)₂NHR^{Z}, —NHS(O)₂R^{Z}, —NR^{Z}₂, —NHR^{Z} and —OR^{Z};
each R^{Z} independently represents C1-10 alkyl, C2-10 alkenyl, C6-10 aryl, or C4-10 heterocyclyl;
or an N-oxide thereof or a pharmaceutically acceptable salt thereof or a pharmaceutically acceptable solvate thereof.

Surprisingly, the present inventors have determined that compounds of Formula (I) exhibit broad antiviral activity, especially against mRNA viruses. These compounds contain only one hydrocarbon group. Preferably the hydrocarbon group is a C1-C30 alkyl group or a C2-C30 alkenyl group.

The present inventors have surprisingly determined that the class of compounds of Formula (I) inhibit eIF4A. Therefore, the compounds of Formula (I) can be used to treat diseases that are sensitive to the inhibition of eIF4A. This includes other conditions such as CNS-related disorders, such as Alzheimer's disease, Parkinson's disease, Huntingdon's disease, muscle wasting, autism spectrum disorder, frontotemporal dementia and motor neurone disease.

One example of a disease that is treated by the inhibition of eIF4A is viral infections. In this regard, the present inventors have shown that the compounds of Formula (I) exhibit broad spectrum antiviral activity.

In particular, the present inventors have specifically shown that the claimed compounds can be used to treat viruses including the influenza viruses H1N1, H2N3, LPAI H2N3, Zika virus and Schmallenberg orthobunyavirus. This illustrates the breadth of utility of the compounds of Formula (I) as antiviral treatments.

As well as being effective against a wide variety of viruses, the compounds of Formula (I) have been demonstrated to be resilient against the evolution of antiviral resistance in viruses.

The prior art includes general disclosures relating to the use of galactolipids similar to those defined by the claimed invention in relation to anti-cancer, anti-obesity and anti-inflammatory treatments.

However, the present invention provides distinct advantages relating to the inhibition of eIF4A or the treatment of specific conditions and diseases using such compounds.

Specifically, the documents Luyen et al., Arch. Pharm. Res. (2015) 38:1011-1018, Yokosuka et al., Natural Product Communications (2013) 8(3), 315-318, Kiem et al., Arch Pharm Res (2012), 35(12), 2135-2142, Nagatsu et al., Bioorg. Med. Chem. Lett., 1994, 4(13), 1619-1622, Wang et al., Natural Product Research 2020, 34(3), 351-358, CN106995475A and U.S. Pat. No. 6,531,582B1 make no mention of the inhibition of eIF4A or the prevention or treatment of eIF4A-related conditions or disorders such as viral infections and CNS-related disorders.

In addition, these documents do not disclose, or contain any teaching towards, the preferred compounds of the claimed invention, particularly those including a disaccharide group and/or a C15-C17 hydrocarbon group.

The present invention provides significant advances in terms of the use of the compounds of the invention for inhibiting eIF4a, especially for treating viral infections, or for treating CNS-related disorders such as Alzheimer's disease, Parkinson's disease, Huntingdon's disease, frontotemporal dementia, motor neurone disease, muscle wasting and autism spectrum disorder.

It is preferred that R¹ represents a disaccharide as this can enhance cell entry and improve ADME properties, for example lowering the log P of the compound, whilst retaining or enhancing eIF4A inhibitory activity. Thus, these compounds may provide enhanced treatments as antivirals and in the treatment of CNS-related disorders, for example.

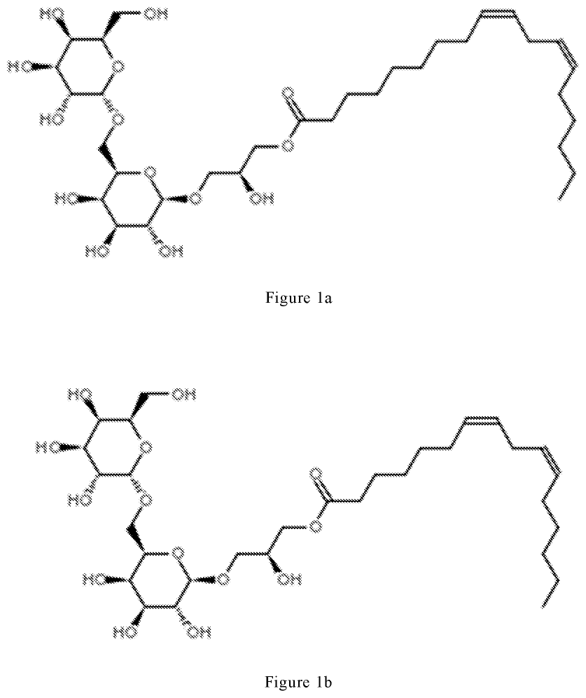

The compound of Formula (I) is preferably digalactosylmonoacylglycerol (DGMG), for example DGMG C18:2 as shown in FIG. 1a of the accompanying drawings, or DGMG 16:2 as shown in FIG. 1b of the accompanying drawings. DGMG is naturally produced by plants such as tomato plants. DGMG is reported to be a stress response molecule in plants. The synthesis of DGMG is elevated in Arabidopsis in response to heat stress.

To date, there have been no reports of the use of DGMG, and related compounds, as medicaments, or the effects of supplementation or treatment with such compounds on human or animal health.

However, as shown by the Examples, the present inventors have surprisingly determined that DGMG is an effective inhibitor of eIF4A. As discussed above, inhibition of eIF4A can be employed to treat a variety of conditions.

Compounds of Formula (I), such as DGMG, can show prolonged and sustained activity after 24 hours and to at least 48 hours post-infection. This indicates that treatment of viral infections with compounds of Formula (I) not be affected by the virus altering its normal mechanism of action to circumvent the action of the compound of Formula (I).

According to a second aspect the present invention provides a compound of Formula (I) for use in the treatment or prevention of a disease or condition which is caused by dysregulation of protein translation, and/or for use as an inhibitor of protein translation, a chemotherapeutic agent, a cell sensitising agent, an antiproliferative agent, an antiviral agent or an adjuvant, and/or for use in the treatment or prevention of disease or condition selected from the group consisting of: viral infection, cancer and CNS-related disorders. The compound may be used in a method of treatment or prevention comprising administering an eIF4A inhibitor selected from the list consisting of: elatol, hippuristanol, zotatifin, pateamine A, CR-1-31-B and silvestrol.

According to a third aspect the present invention provides a composition for use as a medicament, wherein the composition comprises a compound of Formula (I). The composition may be an extract of a plant or fruit thereof.

According to a fourth aspect the present invention provides a composition comprising a compound of Formula (I) for use in the treatment of a disease or condition which is caused by dysregulation of protein translation, and/or for use as an inhibitor of protein translation, a chemotherapeutic agent, a cell sensitising agent, an antiproliferative agent, an antiviral agent or an adjuvant, wherein the composition, and/or for use in the treatment of disease or condition selected from the group consisting of: viral infection, cancer, and CNS-related disorders.

According to a fifth aspect the present invention provides a pharmaceutical composition comprising: a compound of formula (I), or a pharmaceutically acceptable salt thereof or a pharmaceutically acceptable solvate thereof; and a pharmaceutically acceptable carrier, diluent or excipient. The pharmaceutical composition may be for use as a medicament, for example for use in the treatment or prevention of a disease or condition which is caused by dysregulation of protein translation, and/or for use as an inhibitor of protein translation, a chemotherapeutic agent, a cell sensitising agent, an antiproliferative agent, an antiviral agent or an adjuvant, wherein the composition, and/or for use in the treatment or prevention of disease or condition selected from the group consisting of: viral infection, cancer, and CNS-related disorders.

According to a sixth aspect the present invention provides a compound for use in a method of treatment or prevention, wherein the compound is an anti-cancer agent and/or an eIF4A inhibitor selected from the list consisting of: elatol, hippuristanol, zotatifin, pateamine A, CR-1-31-B and silvestrol, and wherein the method comprises administering the eIF4A inhibitor and administering a compound of Formula (I) or a composition thereof.

According to a seventh aspect the present invention provides a kit comprising:
a first therapeutic agent comprising a compound of Formula (I) or a composition thereof, and
a second therapeutic agent comprising an anti-cancer agent,
wherein the anti-cancer agent is provided in a form suitable for, and/or with instructions for, administration in a daily dosage which is significantly reduced (e.g. by 10% or more, or 20% or more, or 30% or more) compared to the dosage of the anti-cancer agent if administered alone. The first and second therapeutic agents may be intended to be administered simultaneously, sequentially or separately. The anti-cancer agent may be a chemotherapeutic agent.

The compounds of Formula (I), and compositions thereof, can therefore be used in a method of treatment or prevention of the human or animal body by therapy. The present disclosure also provides a method of treating (e.g. as a preventative) a subject comprising administering a compound of Formula (I), or a composition thereof, to a subject. The method may involve using the compound of Formula (I), or a composition thereof, as an antiviral agent, a protein translation inhibitor, a chemotherapeutic agent, a cell sensitising agent, an antiproliferative agent or an adjuvant, and/or treating a condition selected from the list of: viral infection, cancer, and CNS-related disorders.

As discussed above, viruses such as influenza viruses result in substantial agronomic loss when they infect livestock. Supplementation with compounds to reduce severity of infection can therefore mitigate loss and improve yield.

The present inventors have identified that the impact of such an infection can be alleviated using the compounds of the present invention. Even if a treatment does not completely prevent cure the viral infection, or completely remove symptoms, a reduction in the severity of the viral infection and/or symptoms would be beneficial.

It has been shown that compounds of the invention can be used to significantly increase the yield or appearance of an animal or herd of animals.

The compounds of Formula (I), and compositions thereof, may also therefore be used as a feedstock for an animal or herd of animals, or as part of such feedstock. It has also been rationalised that the compounds of Formula (I), and compositions thereof, can be used to increase the yield of an animal or a herd of animals.

The compounds of Formula (I), and compositions thereof, may also be used to improve the appearance of the animal or herd thereof. The use of the compounds of Formula (I) and compositions thereof may, therefore, be non-therapeutic. For example, the use of compounds of Formula (I) and compositions thereof may be solely cosmetic.

According to an eighth aspect, the present invention provides a use of a compound of Formula (I), or a composition thereof, to increase the yield of an animal or herd of animals, or to improve the appearance of the animal or herd thereof.

According to a ninth aspect, the present invention provides a nutraceutical composition comprising a compound of formula (I), as defined in relation to Formula (I), or a pharmaceutically acceptable salt thereof or a pharmaceutically acceptable solvate thereof. The nutraceutical composition may be a feed composition, preferably an animal feed.

### DETAILED DESCRIPTION

The present invention relates to compounds of Formula (I). It has been determined that compounds of Formula (I) are inhibitors of eIF4A. Formula (I) includes tautomeric or stereochemically isomeric forms thereof, an N-oxide thereof, a pharmaceutically acceptable salt thereof, and/or a pharmaceutically acceptable solvate thereof. It will be appreciated that a disclosure made in relation to one aspect of the present invention may equally relate to other aspects of the present invention. For example, a disclosure made in relation to the compound of first aspect, or Formula (I), or an embodiment thereof, may equally apply to compositions thereof, unless otherwise stated or incompatible.
As used herein, the term “carbohydrate” refers to a compound comprising carbon atoms, hydrogen atoms and oxygen atoms. A carbohydrate group can comprise atoms in addition to carbon, hydrogen and oxygen, but will contain at least these types of atoms. The term “carbohydrate” encompasses both cyclized and open chain forms of a compound comprising carbon, hydrogen and oxygen. Thus compounds comprising open chains, such as sorbitol and mannitol, are also encompassed by the term “carbohydrate”. However, cyclic carbohydrates are preferred. The term “carbohydrate” is intended to be used in its broadest sense to cover sugars and saccharides, such as monosaccharides, disaccharides, oligosaccharides and polysaccharides. Examples of carbohydrate groups include, but are not limited to, D-arabinose, L-arabinose, D-ribose, L-ribose, D-xylose, L-xylose, D-glucose, L-glucose, D-fructose, L-fructose, D-galactose, L-galactose, D-mannose, L-mannose, D-altrose, L-altrose, D-allose, L-allose, D-gulose L-gulose, D-idose, L-idose, D-talose, L-talose, D-sucrose, L-sucrose and D-lactose.
As used herein, the term “hydroxyl” refers to —OH.
As used herein, the term “alkyl” refers to a saturated straight-chain or branched-chain alkyl group. Examples of alkyl groups include methyl, ethyl, n-propyl, isopropyl, n-butyl, isobutyl, sec-butyl, tert-butyl, pentyl, isopentyl, neo-pentyl, iso-amyl, hexyl, heptyl. octyl, and nonyl.
As used herein, the term “alkenyl” refers to an unsaturated straight-chain or branched-chain hydrocarbon group having at least one carbon-carbon double bonds. Examples of alkenyl groups include decenyl, dodecenyl, undecenyl, tridecenyl, tetradecenyl, pentadecenyl, hexadecenyl, heptadecenyl, octadecenyl and nonadecenyl.
As used herein, the term “alkynyl” refers to an unsaturated straight-chain or branched-chain hydrocarbon group having one or more carbon-carbon triple bonds.
As used herein, the term “amino” refers to —NRR′, wherein R and R′ are independently selected from the group consisting of hydrogen, alkyl, heteroalkyl, aryl, carbocyclyl, and heterocyclyl, or where R and R′ may be combined to form a heterocyclyl group. Preferably the R and R′ have from 0 to 6 carbon atoms, such as from 0 to 4 carbon atoms, e.g. 0 or 1 or 2 carbon atoms.
As used herein, the term “amido” refers to —N(COR)R′, wherein R and R′ are independently selected from the group consisting of hydrogen, alkyl, heteroalkyl, aryl, carbocyclyl, and heterocyclyl. Preferably the R and R′ have from 0 to 6 carbon atoms, such as from 0 to 4 carbon atoms, e.g. 0 or 1 or 2 carbon atoms.
As used herein, the term “carbonyl linking group” refers to a —C(═O)— or —C(═O)—R″— group, wherein R″ is selected from the group consisting of alkyl, heteroalkyl, aryl, carbocyclyl, and heterocyclyl. Preferably R″ has from 1 to 6 carbon atoms, such as from 1 to 4 carbon atoms, e.g. 1 or 2 carbon atoms.
As used herein, the term “C-ester linking group” refers to a —C(═O)O— or —C(═O)O—R″— group, wherein R″ is selected from the group consisting of alkyl, alkenyl, heteroalkyl, aryl, carbocyclyl, and heterocyclyl. Preferably R″ has from 1 to 6 carbon atoms, such as from 1 to 4 carbon atoms, e.g. 1 or 2 carbon atoms.
As used herein, the term “O-ester linking group” refers to an —OC(═O)— or an —OC(═O)R″— group, wherein R″ is selected from the group consisting of alkyl, alkenyl, heteroalkyl, aryl, carbocyclyl, and heterocyclyl. Preferably R″ has from 1 to 6 carbon atoms, such as from 1 to 4 carbon atoms, e.g. 1 or 2 carbon atoms.
As used herein, the term “C-amido linking group” refers to a —C(═O)NH— or —C(═O)NR″— group, wherein R″ is selected from the group consisting of alkyl, alkenyl, heteroalkyl, aryl, carbocyclyl, and heterocyclyl. Preferably R″ has from 1 to 6 carbon atoms, such as from 1 to 4 carbon atoms, e.g. 1 or 2 carbon atoms.
As used herein, the term “N-amido linking group” refers to an —NHC(═O)— or an —NR″C(═O) group, wherein R″ is selected from the group consisting of alkyl, alkenyl, heteroalkyl, aryl, carbocyclyl, and heterocyclyl. Preferably R″ has from 1 to 6 carbon atoms, such as from 1 to 4 carbon atoms, e.g. 1 or 2 carbon atoms.
As used herein, the term “ether linking group” refers to a —O—, —O—R″—, —R″—O— or —R″—O—R″— group, wherein R″ is selected from the group consisting of alkyl, alkenyl, heteroalkyl, aryl, carbocyclyl, and heterocyclyl. Preferably R″ has from 1 to 6 carbon atoms, such as from 1 to 4 carbon atoms, e.g. 1 or 2 carbon atoms.
As used herein, the term “thioether linking group” refers to a —S—, —S—R″—, —R″—S— or —R″—S—R″— group, wherein R″ is selected from the group consisting of alkyl, alkenyl, heteroalkyl, aryl, carbocyclyl, and heterocyclyl. Preferably R″ has from 1 to 6 carbon atoms, such as from 1 to 4 carbon atoms, e.g. 1 or 2 carbon atoms.

### R¹

R¹ represents a group selected from the list of: a carbohydrate group or a derivative thereof, a C1-C30 alkyl group or a derivative thereof, a C2-C30 alkenyl group or a derivative thereof, and a C2-C30 alkynyl group or a derivative thereof.

R¹ may be selected from a carbohydrate group or derivative thereof, or a C10-C24 alkyl, alkenyl or alkynyl group or a derivative thereof.

Preferably R¹ represents a carbohydrate group or a derivative thereof, such as a sugar group or a derivative thereof. This group may be bound to the rest of the molecule via a glycosidic bond. The carbohydrate group may suitably be an α-glycoside or a β-glycoside. However, it is not essential that the sugar group (or derivative thereof) is bound to the rest of the molecule via a glycosidic bond. In one embodiment, the compound does not include a glycoside linkage. This can result in a product that is degraded more slowly as it does not have an anomeric position at which it can be readily cleaved. A product that is more difficult to cleave enzymatically will be more stable. Options for the linking group X¹ are set out below and it will be seen that these include linking groups such as alkylene groups which are therefore non-glycosidic. The carbohydrate group may be an L-stereoisomer or a D-stereoisomer.

The carbohydrate group may be a monosaccharide, disaccharide, oligosaccharide, or polysaccharide. Preferably the carbohydrate group is a monosaccharide or, more preferably, a disaccharide, or a derivative thereof. A disaccharide (also called a double sugar or biose) is the sugar formed when two monosaccharides are joined directly together by a glycosidic bond. The carbohydrate group or derivative thereof may be selected from the list consisting of allose, altrose, glucose, mannose, gulose, idose, galactose and talose, or a derivative thereof.

Preferably the R¹ group has at least 3 carbon atoms, or at least 4 carbon atoms, e.g. from 4-26 carbon atoms or from 5-24 carbon atoms or from 6-20 carbon atoms. In one preferred embodiment the R¹ group has at least 6 carbon atoms, e.g. from 6-24 carbon atoms.

The carbohydrate group may be unprotected or protected; in other words it may have all of its hydroxyl groups in free form, or some or all of the hydroxyl groups may have been converted to be in protected form. Protecting groups for the hydroxyl groups of a carbohydrate are well known in the art and include, but are not limited to, esters, ethers and silylethers. For example, ether protecting groups may include methyl ether, trityl ether, triphenylmethyl ether, methoxymethyl ether, benzyl ether, p-methoxybenzyl ether and tetrahydropyranyl ether. Silyl ether protecting groups may include ethers based on trimethylsilyl, triethylsilyl, triisopropylsilyl, t-butyldimethylsilyl and t-butyldiphenylsilyl. Ester protecting groups may include trifluoroacetyl ester, acetyl ester, trimethylacetyl ester and benzoyl ester.

In some derivatives of a carbohydrate group adjacent hydroxyl groups can be linked via ester linkages, such as O—R—O, where R is alkylene, e.g. C1-C6 alkylene, such as methylene or iso-propylene. In some embodiments there are two pairs of adjacent hydroxyl groups linked in this manner. Thus the carbohydrate group may contain one or more (e.g. two or more) of the hydroxyl groups in protected form. It may be that all of the hydroxyl groups are in protected form. Where more than one hydroxyl group is protected, the protecting groups may be the same or may be different. It may be that all of the axially oriented hydroxyl groups are protected and/or it may be that all of the equatorially oriented hydroxyl groups are protected. The carbohydrate group may have one or more of its hydroxyl groups protected by acetyl ester protecting groups and/or benzyl ether protecting groups. The carbohydrate group may have all of its hydroxyl groups protected by acetyl ester protecting groups, benzyl ether protecting groups, or a combination thereof. The carbohydrate group may have all of its hydroxyl groups protected by acetyl ester protecting groups.

The carbohydrate group may encompass one or more (e.g. two or more) of the hydroxyl groups being converted to amido or amino groups. It may be that only one or two of the hydroxyl groups are converted to amido or amino groups. It may be that all of the hydroxyl groups are converted to amido or amino groups. Where more than one hydroxyl group is converted, the amido or amino groups to which they are each converted may be the same or may be different. Examples of amino and amido groups include, but are not limited to, —NH₂, —NHMe, —NMe₂, —N(COMe)H, —N(COEt)H, and —N(COMe)Me. In general, it may be that the derivative of a carbohydrate group is one where one or more (e.g. two or more) of the hydroxyl groups have been converted to a nitrogen containing functional group, such as an azide or an amine or an amide group. Benefits of this derivatisation are that the compound can then be immobilised for conducting protein pull-down experiments. One or more (e.g. two or more) of the hydroxyl groups may have been converted to alkyl groups, e.g. C1-C6 alkyl, such as methyl or ethyl groups.

The carbohydrate may comprise a sugar (e.g. a cyclic sugar) with six carbon atoms and the hydroxyl group that is at the C6 position is modified such that there is no longer a free —OH at that position, e.g. due to the hydroxyl group having been converted to an alkyl group or to a nitrogen containing functional group, such as an azide or an amine or an amide group; this group may, for example, have up to 3 carbon atoms, such as 0, 1 or 2 carbon atoms. The hydroxyl group at C6 may be converted to an azide or an amine group.

The carbohydrate group (or the derivative thereof) is preferably cyclic, i.e. include one or cyclic carbohydrate unit. However, it may optionally be linear. The carbohydrate group may have any suitable number of atoms in its ring, for example 3, 4, 5, 6 or 7; preferably 4, 5 or 6. The carbohydrate group may have any suitable number of carbons, for example 3, 4, 5, 6 or 7; preferably 4, 5 or 6. The carbohydrate group may include, or be, a hexose. The carbohydrate group may include, or be, a pentose or a heptose. The carbohydrate group may include, or be, a tetrose.

The disaccharide group may be represented by the formula:

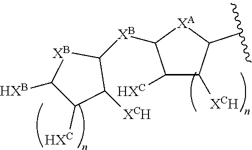

wherein:
each X^{A} group independently represents —CH₂—, —O—, —S—, —NH— or —PH—;
each X^{B} group independently represents —O—, —S—, —NH—, —PH—, or —(CH₂)ₘX^{C}—;
each X^{C} group independently represents —O—, —S—, —NH—, or —PH—;
each n represents 1 or 2;
each m represents 1 or 2; and
the disaccharide group is optionally substituted with one or more Y group.

The monosaccharide group may be represented by the formula:

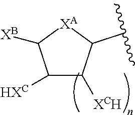

wherein:
X^{A} represents —CH₂—, —O—, —S—, —NH— or —PH—;
X^{B} represents —O—, —S—, —NH—, —PH—, or —(CH₂)ₘX^{C}—;
each X^{C} group independently represents —O—, —S—, —NH—, or —PH—;
each n represents 1 or 2;
each m represents 1 or 2; and
the monosaccharide group is optionally substituted with one or more Y group.

Preferably each X^{A} group of the above monosaccharide and/or disaccharide independently represents —O— or —S—, most preferably —O—.

For the X^{B} groups of the above monosaccharide and/or disaccharide, preferably the heteroatom is bonded to the hydrogen atom, such that each —X^{B}H independently represents —OH, —SH, —NH₂, —PH₂, or —(CH₂)ₘX^{C}H. Preferably each X^{B} group independently represents —O—, —S—, —(CH₂)ₘO—, or —(CH₂)ₘS—. More preferably each X^{B} group independently represents —CH₂O—, or —CH₂S—; most preferably —CH₂O—.

Preferably each X^{C} group of the above monosaccharide and/or disaccharide independently represents —O— or —S—, most preferably —O—.

n of the above monosaccharide and/or disaccharide represents the number of —CH(X^{C}H)— groups present in the brackets shown in the formula above. Preferably each n is 2. Where n is 1 a five-membered ring is present; where n is 2 a six-membered ring is present. Preferably both n are 2; in this embodiment the structure can also be represented as:

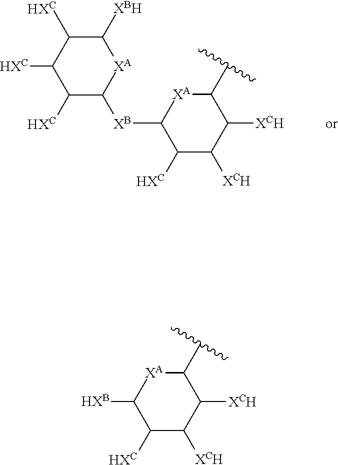

The disaccharide is optionally substituted with one or more Y group, for example from one to five Y groups, or from one to three Y groups. Y groups for the disaccharide group are as disclosed below in relation to Formula (I). This substitution may be present in the place of any one or more hydrogen atoms in the disaccharide group. The disaccharide group may be substituted with 0, 1 or 2 Y groups. Preferably the disaccharide group not substituted with any Y groups.

It may be that each X^{A} group independently represents —O— or —S—, and each X^{B} group independently represents —O—, —S—, —(CH₂)ₘO—, or —(CH₂)ₘS—, and each X^{C} group independently represents —O— or —S—, and n is 1 or 2, and the disaccharide group is substituted by from 0 to 2 Y groups. Preferably, each X^{A} group independently represents —O—, and each X^{B} group independently represents —(CH₂)ₘO—, or —(CH₂)ₘS—, and each X^{C} group independently represents —O—, and n is 2, and the disaccharide group is not substituted by any Y groups.

The carbohydrate group may be a disaccharide group and preferably consists of two monosaccharides that are pentose and/or hexose monosaccharides.

The carbohydrate group may consist of one or two saccharide groups selected from the list consisting of: fructose, galactose, glucose, mannose ribose and deoxyribose, for example from the list consisting of: galactose, glucose and mannose. R¹ may be a galactoside or a glucoside, or a derivative thereof, in other words the sugar group is galactose or glucose. However, it could be other glycosides, such as a fructoside or a glucuronide, or derivatives thereof. Preferably the carbohydrate group comprises at least one galactose group. A galactose sugar group or derivative thereof may be preferred in some embodiments. A glucose sugar group or derivative thereof, or a mannose sugar group or derivative thereof, may be preferred in other embodiments. Increased activity may be seen with these groups. In one embodiment, the carbohydrate group comprises a glucose sugar group or derivative thereof. Preferably the disaccharide group is not functionalised other than with alcohol and ether groups (in the form of acetal or ketal groups). The R¹ group may be an alpha-D-glucosidyl and/or a beta-D-glucosidyl group. It may alternatively be an alpha-L-glucosidyl and/or a beta-L-glucosidyl group. The R¹ group may be an alpha-D-galactosidyl and/or a beta-D-galactosidyl group. It may alternatively be an alpha-L-galactosidyl and/or a beta-L-galactosidyl. The R¹ group may be an alpha-D-mannosidyl and/or a beta-D-mannosidyl group. It may alternatively be an alpha-L-mannosidyl and/or a beta-L-mannosidyl.

More preferably the monosaccharide group is a galactosyl group, for example wherein the galactosyl group is represented by the formula:

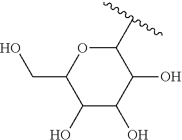 [SMILES: *C1OC(CO)C(O)C(O)C1O]

Yet more preferably the galactosyl group is represented by the formula immediately below this paragraph. In one embodiment any of the above-disclosed formulae for the monosaccharide group have the stereochemistry shown by the formula:

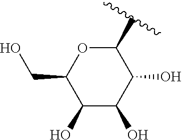 [SMILES: *[C@@H]1O[C@H](CO)[C@H](O)[C@H](O)[C@H]1O]

It is most preferable that the carbohydrate group is a disaccharide group. More preferably the disaccharide group is a digalactosyl group, for example wherein the digalactosyl group is represented by the formula:

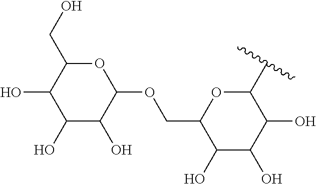 [SMILES: *C1OC(COC2OC(CO)C(O)C(O)C2O)C(O)C(O)C1O]

Yet more preferably the digalactosyl group is represented by the formula immediately below this paragraph. In one embodiment any of the above-disclosed formulae for the disaccharide group have the stereochemistry shown by the formula:

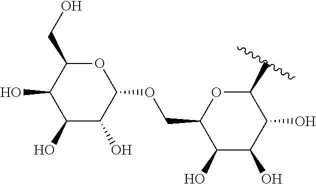 [SMILES: *[C@@H]1O[C@H](CO[C@H]2O[C@H](CO)[C@H](O)[C@H](O)[C@H]2O)[C@H](O)[C@H](O)[C@H]1O]

The carbohydrate group may be a C5-C30 group, such as a C5-C26 or C5-C22 group. Preferably the carbohydrate group is a C5-C18 group, such as a C5-C16 group, or a C5-C14 group. It is also preferable for the carbohydrate group to be a C10-C22 group, such as a C12-C18 or a C12-C16 group, for example a C12-C14 group. More preferably the carbohydrate group is a C11-C13 group, most preferably a C12 group, or a C5-7 group, most preferably a C6 group.

The disaccharide group may include one or more —OH group, for example two or three or more —OH groups. Preferably the disaccharide group includes four or five or more —OH groups, more preferably six or seven. For example, the disaccharide group may include from 1 to 10 —OH groups, or from 3 to 10 —OH groups, preferably from 5 to 9 —OH groups, for example from 6 to 8 —OH groups.

It will be understood that the denomination “C[number]” in relation to a group defines the number of carbon atoms in the group. The denomination “C[number 1]-C[number 2]” in relation to a group defines that the number of carbon atoms in the group ranges from [number 1] to [number 2]. For example, “C6-C30 alkyl” represents an alkyl group containing from 6 to 30 carbon atoms.

R² and R³

A hydrocarbon group is selected from a C1-C30 alkyl group or a derivative thereof, a C2-C30 alkenyl group or a derivative thereof, and a C2-C30 alkynyl group or a derivative thereof. n is 0 or 1. Where n is 0, R³ represents the hydrocarbon group. Where n is 1, either: a) R² represents H or R^{Z} and R³ represents the hydrocarbon group, or b) R² represents the hydrocarbon group and R³ represents H or R^{Z}.

In other words, where n is 1, and R² is therefore present, one of R² and R³ represents the hydrocarbon group, and the other of R² and R³ represents H.

The hydrocarbon group may represent C6-C30 alkyl or C6-C30 alkenyl. Optionally the hydrocarbon group includes one or more Y group.

It will be understood that the carbon atoms of the Y group are included in the required range of carbon atoms for the hydrocarbon group. Thus, the total number of carbon atoms for the R² and R³ groups, including any Y group(s), is no less than 6 and no more than 30.

The alkyl, alkenyl or alkynyl group may be straight chain or branched; preferably it is straight chain.

The hydrocarbon group may be selected from a C1-C24 alkyl or a C1-C24 derivative of an alkyl group, a C2-C24 alkenyl or a C2-C24 derivative of an alkenyl group, and a C2-C24 alkynyl group or a C2-C24 derivative of an alkynyl group. The hydrocarbon group may be selected from a C2-C24 alkyl group or a derivative thereof, a C2-C24 alkenyl group or a derivative thereof and a C2-C24 alkynyl group or a derivative thereof, or it may be selected from a C6-C24 alkyl group or a derivative thereof, a C6-C24 alkenyl group or a derivative thereof and a C6-C24 alkynyl group or a derivative thereof. The hydrocarbon group is preferably a C10-C24 alkyl, alkenyl or alkynyl group, or a derivative thereof. Preferably, the hydrocarbon group is a C10-C20 alkyl, alkenyl or alkynyl group, or derivative thereof, such as a C10-C18 or a C12-C18 alkyl, alkenyl or alkynyl group, or derivative thereof. It may, for example, be a C12-C24 group, a C12-C20 group or a C13-C20 group or a C14-C20 group. The hydrocarbon group may be a C10-C24 derivative of an alkyl, alkenyl or alkynyl group. Preferably, the hydrocarbon group is a C10-C20 derivative of an alkyl, alkenyl or alkynyl group, such as a C10-C18 or a C12-C18 derivative of an alkyl, alkenyl or alkynyl group. It may be a C12-C24 group, a C12-C20 group or a C13-C20 group. Preferably the hydrocarbon group is a C15-C17 (e.g. C16) group, for example a C15-C17 alkyl or alkenyl group, more preferably a C15-C17 alkenyl group, most preferably a C16 alkenyl group.

The alkyl, alkenyl or alkynyl group need not be the sole provider of the carbon atoms to meet the stated range; carbon atoms may also be contributed by the modification of these groups to form the derivative. This applies in the embodiment described below where the derivative of an alkyl, alkenyl or alkynyl group that is encompassed by the present invention is one where one or more (e.g. two or more) of the hydrogen atoms in the hydrocarbon chain are replaced with substituent groups and where these substituent groups include one or more carbon atoms.

Derivatives of alkyl, alkenyl and/or alkynyl groups can include where one or more (e.g. two or more) of the carbon atoms in the hydrocarbon chain are replaced with heteroatoms. The heteroatom(s) may, for example, be selected from O, N, S, SO, SO₂, P, B, Si, and combinations thereof. For example, the heteroatom(s) may be selected from O, N, S, and combinations thereof. In one embodiment from 1 to 5 carbon atoms in the group are replaced with heteroatom(s), e.g. 1, 2 or 3 carbon atoms in the group might be replaced with heteroatom(s). When more than one carbon atom in the group is replaced, the heteroatoms used may be the same or may be different.

Therefore, for example, the hydrocarbon group may include an ether, amine, thioether, sulfoxide, sulfone, and/or sulfonamide group in the chain. Clearly, the number of carbon atoms in the alkyl, alkenyl or alkynyl group of the hydrocarbon group will be reduced where one or more of the carbon atoms in the hydrocarbon chain are replaced with heteroatoms. However, the skilled person would readily be able to see how many carbon atoms would have been in the hydrocarbon chain had one or more of these not been replaced with heteroatoms.

Derivatives of alkyl, alkenyl and/or alkynyl groups can include where one or more (e.g. two or more) of the hydrogen atoms in the hydrocarbon chain are replaced with substituent groups. In one embodiment from 1 to 10 hydrogen atoms in the group are substituted, such as from 1 to 6, e.g. 1, 2, 3 or 4 of the hydrogen atoms in the hydrocarbon chain might be replaced with substituent groups. When more than one hydrogen atom in the group is replaced, the substituent groups used may be the same or may be different. For example, the alkyl, alkenyl or alkynyl group may be substituted with one or more substituent groups independently selected from hydroxyl and amino and carboxyl groups, and aryl or heteroaryl groups (especially unsaturated cyclic and heterocyclic groups with 5 to 10 atoms (e.g. 6 to 10 atoms) in their ring, such as imidazolyl, thiazolyl, thienyl, phenyl, tolyl, xylyl, pyridinyl, pyrimidinyl, pyrazinyl, indolyl or naphthyl groups). The alkyl, alkenyl or alkynyl group may be substituted with one or more substituent groups independently selected from hydroxyl and amino and carboxyl groups. The alkyl, alkenyl or alkynyl group may be substituted with one or more substituent groups independently selected from aryl or heteroaryl groups, especially unsaturated cyclic and heterocyclic groups with 5 to 10 atoms (e.g. 6 to 10 atoms) in their ring, such as imidazolyl, thiazolyl, thienyl, phenyl, tolyl, xylyl, pyridinyl, pyrimidinyl, pyrazinyl, indolyl or naphthyl groups. The ring itself may be substituted, e.g. with one or more C1-6 alkyl groups, such as one or two (or more) methyl or ethyl groups, as is the case in tolyl and xylyl. Preferably the total number of carbon atoms in each of the substituent groups is from 5 to 12.

The hydrocarbon group may be a substituted alkenyl; for example it may be an (alkyl)-C(H)(OH)-(alkenyl), (alkyl)-C(H)(NH₂)-(alkenyl), (alkenyl)-C(H)(OH)-(alkenyl), or (alkenyl)-C(H)(NH₂)-(alkenyl) group. The total number of carbon atoms in said substituted alkenyl may be from 10-24, such as from 10-20 or 10-18 or 12-18.

The hydrocarbon group may be a substituted alkyl; for example it may be an alkyl group that is substituted with one or more substituent groups that are independently selected from unsaturated cyclic and heterocyclic groups with 5 to 10 atoms (e.g. 6 to 10 atoms) in their ring. Preferably it is an alkyl group that is substituted with two or more substituent groups that are independently selected from unsaturated cyclic and heterocyclic groups with 5 to 10 atoms (e.g. 6 to 10 atoms) in their ring. The substituent groups may be unsaturated cyclic groups with 5 to 10 atoms (e.g. 6 to 10 atoms) in their ring and with a total number of carbon atoms of from 5 to 12, such as phenyl or naphthyl or tolyl or xylyl groups. There may be two substituent groups on the same carbon atom in the alkyl group, and preferably these two substituent groups are the same.

The hydrocarbon group may be a C10-C24 derivative of an alkyl group, where the alkyl group is a C1-12 group and this is substituted with one or more C5-12 substituent groups independently selected from aryl or heteroaryl groups, especially unsaturated cyclic and heterocyclic groups with 5 to 10 atoms (e.g. 6 to 10 atoms) in their ring. Thus the total number of carbon atoms in the hydrocarbon group is C10-C24, and this is made up of carbon atoms from the alkyl group and carbon atoms from the aryl or heteroaryl substituent groups. The hydrocarbon group may be a C10-C20 derivative of an alkyl group, such as a C10-C18 or a C12-C18 derivative of an alkyl group. It may, for example, be a C12-C24 group, a C12-C20 group or a C13-C20 group. It may be that the alkyl group is a C1-8 group and this is substituted with one or more C5-12 substituent groups independently selected from aryl or heteroaryl groups, especially unsaturated cyclic and heterocyclic groups with 5 to 10 atoms (e.g. 6 to 10 atoms) in their ring. Preferably, the alkyl group is a C1-6 group (e.g. C1, C2, C3 or C4) and this is substituted with one or more C5-12 substituent groups independently selected from aryl or heteroaryl groups, especially unsaturated cyclic and heterocyclic groups with 5 to 10 atoms (e.g. 6 to 10 atoms) in their ring. The substituent groups in the derivative of an alkyl group may be selected from unsaturated cyclic groups with 5 to 10 atoms (e.g. 6 to 10 atoms) in their ring, such as phenyl or naphthyl or tolyl or xylyl groups, especially unsaturated cyclic groups with 6 atoms in their ring, such as phenyl or tolyl or xylyl groups.

Preferably the hydrocarbon group is unsubstituted.

One or more (e.g. two or more) of the carbon atoms in the hydrocarbon group may be replaced with heteroatoms and one or more (e.g. two or more) of the hydrogen atoms in the hydrocarbon chain may be replaced with substituent groups. Thus, for example, the hydrocarbon group may include an amide or anhydride group in the chain. The hydrocarbon group may include one or more, for example one or two, carbocyclic and/or heterocyclic rings.

The hydrocarbon group may represent C8-C28 alkyl or C8-C28 alkenyl, such as C10-C26 alkyl or C10-C26 alkenyl, or C12-C26 alkyl or C12-C26 alkenyl. Preferably the hydrocarbon group represents C14-C22 alkyl or C13-C22 alkenyl, such as C13-C20 alkyl or C13-C20 alkenyl. For example, the hydrocarbon group may represent C14-C18 alkyl or C14-C18 alkenyl. Most preferably the hydrocarbon group represents C17 alkyl or C17 alkenyl, or C15 alkyl or C15 alkenyl. Each of the groups described in this paragraph may optionally include one or more Y group.

The hydrocarbon group may be an alkenyl group (or derivative thereof). The hydrocarbon group may be a C10-C24 alkenyl group or a C12-C24 alkenyl group, such as a C12-C20 alkenyl group or a C14-C20 alkenyl group. Preferably the hydrocarbon group includes one or more C═C double bond, for example two or more C═C double bonds, or three or more C═C double bonds. There may be from one to eight C═C double bonds in the hydrocarbon group, such as from one to six C═C double bonds. Preferably the hydrocarbon group is an alkenyl group. The alkenyl group may include one or more alkene groups, such as two or more or three or more. from 1 to 8 alkene groups, or from 1 to 6 alkene groups, such as from 1 to 4 alkene groups, preferably from 1 to 3 alkene groups, for example 2 alkene groups.

More preferably the hydrocarbon group contains 17 carbons and from 0 to 3 alkene groups, or 15 carbons and from 0 to 3 alkene groups. Yet more preferably the X group bonded to the hydrogen represents an ester group, for example wherein the carbon of the ester group is bonded to the hydrocarbon group, and the hydrocarbon group contains 17 carbons and from 0 to 3 alkene groups or 15 carbons and from 0 to 3 alkene groups.

The alkene groups may be at any position in the alkenyl group of the hydrocarbon group. Preferably the alkene group(s) is/are not terminal alkene group(s), i.e. at the very end of the alkenyl group distal from the X²/X³ group. Preferably the alkene group(s) is/are positioned more than two (more preferably three or four or five or more) carbon atoms from the end of the alkenyl chain distal from the X²/X³ group. Preferably the alkene group(s) is/are positioned more than two (more preferably three or four or five or more) carbon atoms from the end of the alkenyl chain proximal to the X²/X³ group.

Preferably each alkene group has a cis geometry. The C═C double bond(s) may be Z-configured (cis) or E-configured (trans). Where there is more than one double bond these may be all are Z-configured, or they may be all E-configured, or there may be combinations of Z-configured and E-configured double bonds. In one embodiment all the C═C double bonds are Z-configured.

Preferably there are one or more, such as from 1 to 6, alkene groups in the alkenyl group of the hydrocarbon group, wherein the alkene groups are positioned more than two carbon atoms (e.g. more than four carbon atoms) from the ends of the alkenyl chain distal from and proximal to the X²/X³ group.

Where there are two or more alkene groups, preferably they are not conjugated. For example, where there are two or more alkene groups, there may be one or more sp³-hybridised carbon atoms between the alkene groups.

For example, there may be from 1 to 3 alkene groups that are each positioned more than two carbons from the ends of the alkenyl chain that are proximal and distal from the X²/X³ groups, and that each have a cis geometry.

Preferably, the hydrocarbon group is a C10-C24 alkenyl and may, e.g., be a straight-chain alkenyl having from 10 to 20 carbon atoms. Preferably, the hydrocarbon group is a C12-C18 alkenyl.

Preferably the hydrocarbon alkenyl group has from one to five C═C double bonds, such as from one to four C═C double bonds, e.g. from one to three C═C double bonds, such as two or three C═C double bonds.

Preferably the hydrocarbon group is an alkenyl group and the (or each) double bond is located at carbon position 5 in the chain or higher, such as position 6 or higher, or position 7 or higher, preferably the (or each) C═C double bond is located at position 8 or higher.

More preferably, the hydrocarbon group is a C14-C18 alkenyl (e.g. a C15, C16 or C17 alkenyl) having one to three C═C double bonds, such as two or three C═C double bonds, for example R² may be a C17 alkenyl having three C═C double bonds.

The alkenyl group may be a 8,11,14-heptadecatrienyl.

### X1

X¹ is a linking group. X¹ may be any linking group provided that this linking group is divalent. Preferably X¹ has from 1-18 carbon atoms, especially from 1-12 carbon atoms, such as from 1-6 carbon atoms, e.g. 1, 2, 3 or 4 carbon atoms. Examples of divalent linking groups include alkylene groups, cycloalkyene groups, alkenylene groups, ether groups, imino groups, carbonyl groups (including ester groups and amido groups and phosphate groups), (hetero)arylene groups, amino groups, thioether groups, and divalent residues containing any of these divalent groups bonded to each other in series. The linking group may optionally be substituted, e.g. with one or more hydroxyl, amino and/or carboxyl groups. The linking group may be a glycoside linking group.

In one embodiment, the compound does not include a glycosidic bond/linkage. This can result in a product that is degraded more slowly as it does not have an anomeric position at which it can be readily cleaved. A product that is more difficult to cleave enzymatically will be more stable. For example, X¹ may be a C1-18 alkylene group, which may optionally be substituted e.g. with one or more hydroxyl, amino and/or carboxyl groups; especially a C1-12 or C1-6 alkylene group e.g. a C1, 2, 3 or 4 alkylene group. For example, alkylene groups are non-glycosidic.

X¹ may contains at least one heteroatom selected from O, P, N and S. At least one such heteroatom may be located in the main chain of the X¹ group, rather than as a branch or substituent group. For example, X¹ may be an ester or an ether or a thioether or an amido or an amino or a phosphate-containing linker group.

X¹ may be selected from the list consisting of: ether (i.e. —O—), thioether (i.e. —S—), amine (i.e. —N(H)—), phosphine (i.e. —P(H)—), ester (i.e. —C(O)O— or —OC(O)—), thioester (i.e. —C(O)S— or —SC(O)—), amide (i.e. —C(O)N(H)— or —N(H)C(O)—), urea (i.e. —N(H)C(O)N(H)—), thiourea (i.e. —N(H)C(S)N(H)—), carbamate (i.e. —OC(O)N(H)— or —N(H)C(O)O—), phosphate (i.e. —OP(O)₂O—) and sulfonamide (i.e. —S(O)₂N(H)— or —N(H)S(O)₂—). X¹ may be selected from the list consisting of: —OCH₂—, —O(CH₂)₂—, —O(CH₂)₃—, —OC(H)(OH)—, —OC(H)(OH)CH₂—, —OC(H)(NH₂)—, —OC(H)(NH₂)CH₂—, —(CH₂)C(O)OCH₂—, —SCH₂—, —CH₂SCH₂—, —OP(O₂)OCH₂—, and —OP(O₂)O(CH₂)₂—.

X¹ may be selected from: (i) a C1-C12 alkylene linking group, e.g. a C1-C8 alkylene linking group, such as methylene or ethylene or propylene or butylene or pentylene; (ii) an ether linking group, such as —(CH₂)ₚO(CH₂)_{q}—, where p and q independently represent an integer of from 0 to 3, e.g. from 1 to 3; (iii) a C2-C6 alkenylene linking group, such as ethenylene; (iv) a carbonyl-containing linker group, especially an ester linking group, such as —(CH₂)ₚCOO(CH₂)_{q}—, or —(CH₂)ₚOC(═O)(CH₂)_{q}—, where p and q independently represent an integer of from 0 to 3, e.g. from 1 to 3; (v) a (hetero)arylene linker, such as —(CH₂)ₚ(Ar)(CH₂)_{q}—, where p and q independently represent an integer of from 0 to 3, e.g. from 1 to 3, and Ar is a C6-C8 arylene substituent group, such as phenylene, or a 5 to 8 membered ring hetero arylene substituent group, such as furylene, thiophenylene or pyridylene; (vi) an amine linker of formula —RxN(Rz)Ry-, for example wherein Rx and Ry are independently C1-C4 alkylene and Rz is H or C1-C4 alkyl, such as —CH₂N(CH₃)CH₂—; (vii) a thioether linker, such as —(CH₂)ₚS(CH₂)_{q}—, where p and q independently represent an integer of from 0 to 3, e.g. from 1 to 3; (viii) a glycoside linker, such as X—R4 group, wherein R4 is a C1-C12 alkyl, cycloalkyl, alkenyl or alkynyl group and X is —O—, —PR^{a}—, —NR^{a}—, —S— or —CR^{a}R^{b}—, wherein R^{a} and R^{b} are independently selected from the group consisting of hydrogen and C1-C4 alkyl.

X¹ may be selected from: (i) a C1-C6 alkylene linking group, e.g. a C1-C5 alkylene linking group, such as methylene or ethylene; (ii) an ether linking group, such as —(CH₂)ₚO(CH₂)_{q}—, where p and q independently represent an integer of from 1 to 3, and p+q equals 4 or less; (iii) a C2-C4 alkenylene linking group such as ethenylene; (iv) an ester linking group —(CH₂)ₚCOO(CH₂)_{q}—, or —(CH₂)ₚOC(═O)(CH₂)_{q}—, where p and q independently represent an integer of from 1 to 3, and p+q equals 4 or less; (v) an amine linker of formula —RxN(Rz)Ry-, for example wherein Rx and Ry are C1-C4 alkylene, e.g. C1 or C2 alkylene, and Rz is H or C1-C4 alkyl, e.g. C1 or C2 alkyl; (vi) a thioether linker such as —(CH₂)ₚS(CH₂)_{q}—, where p and q independently represent an integer of from 1 to 3, and p+q equals 4 or less; (vii) a glycoside linker, such as X—R4 group, wherein R4 is a C1-C12 alkyl, cycloalkyl, alkenyl or alkynyl group and X is —O—, —PR^{a}—, —NR^{a}—, —S— or —CR^{a}R^{b}—, wherein Ra and R^{b} are independently selected from the group consisting of hydrogen and C1-C4 alkyl.

X¹ may be selected from: (i) a C1-C4 alkylene linking group such as methylene or ethylene; (ii) an ether linking group, such as —(CH₂)ₚO(CH₂)_{q}—, where p and q independently represent an integer of from 1 to 3, and p+q equals 4 or less; (iii) a C2-C4 alkenylene linking group such as ethenylene; (iv) an ester linking group —(CH₂)ₚCOO(CH₂)_{q}—, or —(CH₂)ₚOC(═O)(CH₂)_{q}—, where p and q independently represent an integer of from 1 to 3, and p+q equals 4 or less; (v) an amine linker of formula —RxN(Rz)Ry-, for example wherein Rx and Ry are C1-C4 alkylene, e.g. C1 or C2 alkylene, and Rz is H or C1-C4 alkyl, e.g. C1 or C2 alkyl; (vi) a thioether linker such as —(CH₂)ₚS(CH₂)_{q}—, where p and q independently represent an integer of from 1 to 3, and p+q equals 4 or less; (vii) a glycoside linker, such as X—R4 group, wherein R4 is a C1-C12 alkyl, cycloalkyl, alkenyl or alkynyl group and X is —O—, —PR^{a}—, —NR^{a}—, —S— or —CR^{a}R^{b}—, wherein R^{a} and R^{b} are independently selected from the group consisting of hydrogen and C1-C4 alkyl.

X¹ may be selected from the list consisting of: ether, thioether, ester, thioester, amide, urea, thiourea, carbamate, phosphate and sulfonamide. X¹ may be selected from the list consisting of: ether, thioether, ester, thioester, amide, urea, thiourea and carbamate. X¹ may be selected from the list consisting of: ether, thioether, ester, thioester and amide. Preferably, X¹ is an ether or a thioether. Most preferably X¹ is an ether, for example wherein X¹ represents the oxygen atom of a glycosidic bond to the disaccharide group.

Preferably X¹ is a C1-C12 alkylene linking group or a glycoside linker, or an ester linking group having from 1-12 carbon atoms; more preferably a C1-C8 alkylene linking group or a glycoside linker having from 1-8 carbon atoms or an ester linking group having from 1-8 carbon atoms; most preferably a C1-C6 alkylene linking group or a glycoside linker having from 1-6 carbon atoms or an ester linking group having from 1-6 carbon atoms; such as a C1-C4 (e.g. C1 or C2) alkylene linking group or a glycoside linker having from 1-4 carbon atoms (e.g. C1 or C2) or an ester linking group having from 1-4 carbon atoms (e.g. C2 or C3).

Preferably X¹ is a linking group that is a C1-C12 alkylene linking group or a glycoside linker, or an ester linking group, more preferably a C1-C8 alkylene linking group or a glycoside linker. Preferably the alkylene linking groups are straight chain. The linking groups may be branched alkylene groups. For example, X¹ may represent a linking group that is a C1-C12 straight chain alkylene linking group (such as a C1-C8 or C1-C6 straight chain alkylene linking group) or a C2-C12 branched chain alkylene linking group (such as a C2-C8, or C2-C6, or C3-C6 branched chain alkylene linking group).

Preferably X¹ represents a C1-C6 alkylene linking group or a glycoside linker, more preferably a C1-C5 alkylene linking group or a glycoside linker. It may therefore be methylene, ethylene, propylene, butylene or pentylene or a glycoside linker. X¹ may represent a C1-C4 alkylene linking group, such as methylene, ethylene or propylene, or a glycoside linker.

X¹ may be a glycoside linker, such as X—R4 group, wherein R4 is a C1-C12 (e.g. C1-8 or C1-6 or C1-4) alkyl, a C4-C12 (e.g. C4-8 or C4-6) cycloalkyl, C2-C12 (e.g. C2-8 or C2-6 or C2-4) alkenyl, or C2-C12 (e.g. C2-8 or C2-6 or C2-4) alkynyl group and X is —O—, —O(PO₂)O—, —NR^{a}—, —NR^{a}C(═O)—, —PR^{a}—, —S— or —CR^{a}R^{b}—, wherein R^{a} and R^{b} are independently selected from the group consisting of hydrogen and C1-C4 alkyl. Therefore, X¹ may be linked to the rest of the molecule by the group X—R4, wherein X is based on an O, N, S, P or C atom. Thus there may be an O-glycoside bond, a glycosylamine bond, a thioglycoside bond, a P-glycoside bond or a C-glycoside bond. When the group is —NR^{a}—, or —PR^{a}—, R^{a} is selected from the group consisting of hydrogen and C1-C4 alkyl, e.g. it may be hydrogen or methyl. When the group is —CR^{a}R^{b}—, R^{a} and R^{b} are independently selected from the group consisting of hydrogen and C1-C4 alkyl, e.g. each may be hydrogen or methyl. The glycoside linker may be of formula —X—R4-, wherein R4 is a C1-C4 (e.g. C1, 2 or 3) alkyl, a C4-C8 (e.g. C4, 5 or 6) cycloalkyl, or a C2-C6 (e.g. C2, 3 or 4) alkenyl, and X is —O—, —O(PO₂)O—, —NR^{a}—, —S— or —CR^{a}R^{b}—, wherein R^{a} and R^{b} are independently selected from the group consisting of hydrogen and C1-C4 alkyl. The glycoside linker may be of formula —X—R4-, wherein R4 is a C1-C4 (e.g. C1, 2 or 3) alkyl, or a C2-C6 (e.g. C2, 3 or 4) alkenyl, and X is —O—, —O(PO₂)O—, —NR^{a}—, or —S—, wherein R^{a} is selected from the group consisting of hydrogen and C1-C4 alkyl.

Where R¹ is a sugar group, e.g. a monosaccharide or disaccharide, X¹ may link R¹ to the rest of the molecule by an O-glycoside bond. For example, the sugar group of R¹ may be linked to the rest of the molecule by an O—(CH₂)n group, wherein n is an integer of from 1 to 6. n may be 1, 2, 3, 4, 5 or 6. Preferably n is from 1 to 4, e.g. 1, 2 or 3. n may be 1 or 2; preferably n is 1.

Where R¹ is a glucosidyl group preferably X¹ is an —OCH₂— group. However, X¹ could be an —O(CH₂)₂— group or an —O(CH₂)₃— group or an —O(CHOH)— group or an —O(CHNH₂)— group. Alternate linking groups X¹ could be used, as discussed above. Where R¹ is a galactosidyl group preferably X¹ is an —OCH₂— group. However, X¹ could be an —O(CH₂)₂— group or an —O(CH₂)₃— group or an —O(CHOH)— group or an —O(CHNH₂)— group. Alternate linking groups X¹ could be used, as discussed above. Where R¹ is a mannosidyl group preferably X¹ is an —OCH₂— group. However, X¹ could be an —O(CH₂)₂— group or an —O(CH₂)₃— group or an —O(CHOH)— group or an —O(CHNH₂)— group. Alternate linking groups X¹ could be used, as discussed further above.

X² and X³

X² and X³ are the linking groups that are bonded to the R² and R³ groups respectively. Where n=0, R³ represents the hydrocarbon group. Where n=1, one of R² and R³ is the hydrocarbon group and the other of R² and R³ is H.

X² and/or X³ may be any linking group provided that this linking group is divalent. Preferably X² and/or X³ has from 0-18 carbon atoms, especially from 0-12 carbon atoms, such as from 0-6 carbon atoms, e.g. 0, 1, 2, 3 or 4 carbon atoms.

Examples of divalent linking groups include alkylene groups, cycloalkyene groups, alkenylene groups, ether groups, imino groups, carbonyl groups (including ester groups and amido groups and phosphate groups), (hetero)arylene groups, amino groups, thioether groups, and divalent residues containing any of these divalent groups bonded to each other in series. X² and/or X³ may be substituted, e.g. with one or more hydroxyl, amino and/or carboxyl groups.

X² and/or X³ may contain at least one heteroatom selected from O, P, N and S. In one such embodiment at least one such heteroatom is located in the main chain of the linker, rather than as a branch or substituent group. For example, X² and/or X³ may be an ester or an ether or a thioether or an amido or an amino or a phosphate-containing linker group.

Specific examples of X² and/or X³ groups that contain one or more heteroatom, with one or more of the heteroatoms being located in the main chain of the linker, include: —OC(O)—, —OC(O)CH₂—, —N(H)C(O)—, —N(H)C(O)CH₂—, —NCH₂C(O)—, —NCH₂C(O)CH₂—, —OCH₂—, —O(CH₂)₂—, —O(CH₂)₃—, —OC(H)(OH)—, —OC(H)(OH)CH₂—, —OC(H)NH₂—, —OC(H)NH₂CH₂—, —CH)C(O)OCH₂—, —SCH₂—, —CH₂SCH₂—, —OP(O₂)OCH₂—, and —OP(O₂)O(CH₂)₂—.

The X² or X³ group bonded to the hydrocarbon group preferably represents a linker group selected from the list consisting of: ether, thioether, amine, phosphine, ester, thioester, amide, urea, thiourea, carbamate, phosphate and sulfonamide.

The X² or X³ group bonded to the hydrocarbon group is preferably selected from the list consisting of: ether, amine, ester, thioester, amide, urea, thiourea, carbamate, phosphate and sulfonamide. The X² or X³ group bonded to the hydrocarbon group may be selected from the list consisting of: ester, thioester, amide, urea, thiourea, carbamate, phosphate and sulfonamide. Preferably the X² or X³ group bonded to the hydrocarbon group is selected from the list consisting of: ester, amide, urea, and carbamate. More preferably the X² or X³ group bonded to the hydrocarbon group is selected from the list consisting of: ester, amide, urea, and carbamate; especially from the list consisting of: ester and amide.

Most preferably the X² or X³ group bonded to the hydrocarbon group is an ester. The ester may be oriented either way around, i.e. —OC(O)— or —C(O)O—. Preferably the orientation of the ester is —OC(O)R, where R represents the hydrocarbon group. The same consideration is made for the option for the X² or X³ group bonded to the hydrocarbon group being a thioester, amide and sulfonamide.

The X² or X³ group bonded to H is selected from the list consisting of: —O—, —S—, —NH—, —PH—, —C(O)O—, —C(O)NH—, —OP(O)₂O—, and —S(O)₂NH—.

The X² or X³ group bonded to H may be selected from the list consisting of: —O—, —S—, —NH—, —C(O)O—, —C(O)NH—, and —S(O)₂NH—. The X² or X³ group bonded to H may be selected from the list consisting of: —O—, —S—, —NH—. Preferably the X² or X³ group bonded to H is —O— or —S—, most preferably —O—.

The X² or X³ group bonded to the hydrocarbon group may be selected from: (i) a C1-C12 alkylene linking group, e.g. a C1-C8 alkylene linking group, such as methylene or ethylene or propylene or butylene or pentylene; (ii) an ether linking group, such as —(CH₂)ₚO(CH₂)_{q}—, where p and q independently represent an integer of from 0 to 3, e.g. from 1 to 3; (iii) a C2-C6 alkenylene linking group, such as ethenylene; (iv) a carbonyl-containing linker group; especially an ester linking group, such as —(CH₂)ₚC(═O)O(CH₂)_{q}—, or —(CH₂)ₚOC(═O)(CH₂)_{q}—, where p and q independently represent an integer of from 0 to 3, e.g. from 1 to 3, or an amido linking group, such as —(CH₂)ₚNRzC(═O)(CH₂)_{q}—, or —(CH₂)ₚC(═O)NRz(CH₂)_{q}—, where p and q independently represent an integer of from 0 to 3, e.g. from 1 to 3 and Rz is H or C1-C4 alkyl; (v) a (hetero)arylene linker, such as —(CH₂)ₚ(Ar)(CH₂)_{q}—, where p and q independently represent an integer of from 0 to 3, e.g. from 1 to 3, and Ar is a C6-C8 arylene substituent group, such as phenylene, or a 5 to 8 membered ring hetero arylene substituent group, such as furylene, thiophenylene or pyridylene; (vi) an amine linker of formula —RxN(Rz)Ry-, for example wherein Rx and Ry are independently C1-C4 alkylene and Rz is H or C1-C4 alkyl, such as —CH₂N(CH₃)CH₂—; (vii) a thioether linker, such as —(CH₂)ₚS(CH₂)_{q}—, where p and q independently represent an integer of from 0 to 3, e.g. from 1 to 3; (viii) a glycoside linker, such as X—R4 group, wherein R4 is a C1-C12 alkyl, cycloalkyl, alkenyl or alkynyl group and X is —O—, —PR^{a}—, —NR^{a}—, —S— or —CR^{a}R^{b}—, wherein R^{a} and R^{b} are independently selected from the group consisting of hydrogen and C1-C4 alkyl.

The X² or X³ group bonded to the hydrocarbon group may be selected from: (i) a C1-C6 alkylene linking group, e.g. a C1-C5 alkylene linking group, such as methylene or ethylene; (ii) an ether linking group —(CH₂)ₚO(CH₂)_{q}—, where p and q independently represent an integer of from 1 to 3, and p+q equals 4 or less; (iii) a C2-C4 alkenylene linking group such as ethenylene; (iv) an ester linking group —(CH₂)ₚC(O)O(CH₂)_{q}—, where p and q each independently represent an integer of from 0 to 3, and p+q equals 4 or less; or an ester linking group —(CH₂)ₚOC(═O)(CH₂)_{q}—, where p represents an integer of from 1 to 3, q represents an integer of from 0 to 3 and p+q equals 4 or less; or an amido linking group —(CH₂)ₚC(═O)NRz(CH₂)_{q}—, where p and q independently represent an integer of from 0 to 3, and p+q equals 4 or less, and Rz is H or C1-C4 alkyl; or an amido linking group —(CH₂)ₚNRzC(═O)(CH₂)_{q}—, where p represents an integer of from 1 to 3, q represents an integer of from 0 to 3 and p+q equals 4 or less and Rz is H or C1-C4 alkyl; (v) an amine linker of formula —RxN(Rz)Ry-, for example wherein Rx and Ry are C1-C4 alkylene, e.g. C1 or C2 alkylene, and Rz is H or C1-C4 alkyl, e.g. C1 or C2 alkyl; (vi) a thioether linker —(CH₂)ₚS(CH₂)_{q}—, where p represents an integer of from 1 to 3, q represents an integer of from 0 to 3 and p+q equals 4 or less.

The X² or X³ group bonded to the hydrocarbon group may be selected from: (i) a C1-C6 alkylene linking group, e.g. a C1-C5 alkylene linking group, such as methylene or ethylene; (ii) an ether linking group, such as —(CH₂)ₚO(CH₂)_{q}—, where p and q independently represent an integer of from 1 to 3, and p+q equals 4 or less; (iii) a C2-C4 alkenylene linking group such as ethenylene; (iv) an ester linking group such as —(CH₂)ₚC(═O)O(CH₂)_{q}—, or —(CH₂)ₚOC(═O)(CH₂)_{q}—, where p and q each independently represent an integer of from 0 to 3, and p+q equals 4 or less, or an amido linking group, such as —(CH₂)ₚNRzC(═O)(CH₂)_{q}—, or —(CH₂)ₚC(═O)NRz(CH₂)_{q}—, where p and q independently represent an integer of from 0 to 3, and p+q equals 4 or less, and Rz is H or C1-C4 alkyl; (v) an amine linker of formula —RxN(Rz)Ry-, for example wherein Rx and Ry are C1-C4 alkylene, e.g. C1 or C2 alkylene, and Rz is H or C1-C4 alkyl, e.g. C1 or C2 alkyl; (vi) a thioether linker such as —(CH₂)ₚS(CH₂)_{q}—, where p and q independently represent an integer of from 1 to 3, and p+q equals 4 or less.

The X² or X³ group bonded to the hydrocarbon group may be selected from: (i) a C1-C4 alkylene linking group such as methylene or ethylene; (ii) an ether linking group —(CH₂)ₚO(CH₂)_{q}—, where p and q independently represent an integer of from 1 to 3, and p+q equals 4 or less; (iii) a C2-C4 alkenylene linking group such as ethenylene; (iv) an ester linking group —(CH₂)ₚC(═O)O(CH₂)_{q}—, where p and q each independently represent an integer of from 0 to 3, and p+q equals 4 or less; or an ester linking group —(CH₂)ₚOC(═O)(CH₂)_{q}—, where p represents an integer of from 1 to 3, q represents an integer of from 0 to 3 and p+q equals 4 or less; or an amido linking group —(CH₂)ₚC(═O)NRz(CH₂)_{q}—, where p and q independently represent an integer of from 0 to 3, and p+q equals 4 or less, and Rz is H or C1-C3 alkyl; or an amido linking group —(CH₂)ₚNRzC(═O)(CH₂)_{q}—, where p represents an integer of from 1 to 3, q represents an integer of from 0 to 3 and p+q equals 4 or less and Rz is H or C1-C3 alkyl; (v) an amine linker of formula —RxN(Rz)Ry-, for example wherein Rx and Ry are C1-C4 alkylene, e.g. C1 or C2 alkylene, and Rz is H or C1-C4 alkyl, e.g. C1 or C2 alkyl; (vi) a thioether linker —(CH₂)ₚS(CH₂)_{q}—, where p and q independently represent an integer of from 1 to 3, and p+q equals 4 or less.

The X² or X³ group bonded to the hydrocarbon group may be selected from: (i) a C1-C4 alkylene linking group such as methylene or ethylene; (ii) an ether linking group, such as —(CH₂)ₚO(CH₂)_{q}—, where p and q independently represent an integer of from 1 to 3, and p+q equals 4 or less; (iii) a C2-C4 alkenylene linking group such as ethenylene; (iv) an ester linking group such as —(CH₂)ₚC(═O)O(CH₂)_{q}—, or —(CH₂)ₚOC(═O)(CH₂)_{q}—, where p and q each independently represent an integer of from 0 to 3, and p+q equals 4 or less, or an amido linking group, such as —(CH₂)ₚNRzC(═O)(CH₂)_{q}—, or —(CH₂)ₚC(═O)NRz(CH₂)_{q}—, where p and q independently represent an integer of from 0 to 3, and p+q equals 4 or less, and Rz is H or C1-C3 alkyl; (v) an amine linker of formula —RxN(Rz)Ry-, for example wherein Rx and Ry are C1-C4 alkylene, e.g. C1 or C2 alkylene, and Rz is H or C1-C4 alkyl, e.g. C1 or C2 alkyl; (vi) a thioether linker such as —(CH₂)ₚS(CH₂)_{q}—, where p and q independently represent an integer of from 1 to 3, and p+q equals 4 or less.

Preferably X² or X³ group bonded to the hydrocarbon group is a C1-C12 alkylene linking group or a C1-C12 ester linking group, or a C1-12 amido linking group, more preferably a C1-C8 alkylene linking group or a C1-C8 ester linking group or a C1-8 amido linking group. In one embodiment, the alkylene linking groups are straight chain. In another embodiment, the linking groups are branched alkylene groups. For example, X² and/or X³ may represent a linking group that is a C1-C12 straight chain alkylene linking group (such as a C1-C8 or C1-C6 straight chain alkylene linking group) or a C2-C12 branched chain alkylene linking group (such as a C2-C8, or C2-C6, or C3-C6 branched chain alkylene linking group) or a C1-C12 (such as a C1-C8 or C1-C6) ester group or a C1-C12 (such as a C1-C8 or C1-C6) amido group.

Preferably X² or X³ group bonded to the hydrocarbon group is a C1-C6 alkylene linking group or C1-C6 ester group or C1-C6 amido group, more preferably a C1-C5 alkylene linking group or C1-C5 ester group or C1-C5 amido group, such as a C1-C4 alkylene linking group or C1-C4 ester group or C1-C4 amido group. It may therefore be methylene, ethylene, propylene, butylene or pentylene, or an ester linking group —(CH₂)ₚC(═O)O(CH₂)_{q}— or —(CH₂)ₚOC(═O)(CH₂)_{q}—, where p and q each independently represent an integer of from 0 to 3, especially 0, 1 or 2, and p+q equals 4 or less, especially 3 or less, or an amido group —(CH₂)ₚNRzC(═O)(CH₂)_{q}—, or —(CH₂)ₚC(═O)NRz(CH₂)_{q}—, where p and q independently represent an integer of from 0 to 3, especially 0, 1 or 2, and p+q equals 4 or less, especially 3 or less, and Rz is H or C1-C3 alkyl, especially H or C1 alkyl.

The X² or X³ group bonded to the hydrocarbon group may be a C1-C4 alkylene linking group, such as methylene, ethylene or propylene, or an ester linking group —(CH₂)ₚC(═O)O(CH₂)_{q}— or —(CH₂)ₚOC(═O)(CH₂)_{q}—, where p and q each independently represent an integer of from 0 to 3, especially 0, 1 or 2, and p+q equals 3 or less, especially 2 or less, or an amido group —(CH₂)ₚNRzC(═O)(CH₂)_{q}—, or —(CH₂)ₚC(═O)NRz(CH₂)_{q}—, where p and q independently represent an integer of from 0 to 3, especially 0, 1 or 2, and p+q equals 3 or less, especially 2 or less, and Rz is H or C1-C2 alkyl, especially H.

The X² or X³ group bonded to the hydrocarbon group may be an O-ester linking group, C-ester linking group, ether linking group, carbonyl linking group, amine linking group, N-amido linking group, C-amido linking group, thioether linking group or alkylene linking group. X² and/or X³ may be —CH₂—, —CH₂CH₂—, —CH₂CH₂CH₂—, —O—C(═O)—, —NH—, —C(═O)—, —C(═O)—CH₂—, —O—CH₂—C(═O)—, —C(═O)—O—, —NHC(═O)—, —C(═O)NH—, —O—, —CH₂—NH—, —CH₂—NH—CH₂—, —S—, —S—CH₂—, —CH₂—S—CH₂—, or —CH₂—O. X² and/or X³ may be —O—C(═O)—, —NH—, —C(═O)—, —C(═O)—CH₂—, —O—CH₂—C(═O)—, —C(═O)—O—, —NHC(═O)—, —C(═O)NH—, —O—, —CH₂—NH—, —CH₂—NH—CH₂—, —S—, —S—CH₂—, —CH₂—S—CH₂—, or —CH₂—O.

### Z

Each Z group independently represents Y, R^{Z} or H. In one embodiment each Z group independently represents R^{Z} or H. In one such embodiment each R^{Z} may independently represent C1-10 alkyl or C2-10 alkenyl, such as C1-6 alkyl or C2-6 alkenyl, preferably C1-4 alkyl or C2-4 alkenyl. More preferably each Z group independently represents H. Most preferably all Z groups represent H, such that Formula (I) is represented by the structure:

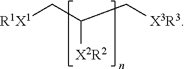

Preferably n=1, such that Formula (I) is represented by the structure:

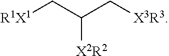

### Y

The hydrocarbon group represents C6-C30 alkyl or C6-C30 alkenyl, optionally including one or more Y group. Each Y group independently represents a group selected from the list consisting of: cyano, halogen, N₃, —C(O)R^{Z}, —C(O)OR^{Z}, —OC(O)R^{Z}, —C(O)NHR^{Z}, —NHC(O)R^{Z}, —NHC(O)NHR^{Z}, —NHC(O)OR^{Z}, —OC(O)NHR^{Z}, —OP(O)₂OR^{Z}, —S(O)₂NHR^{Z}, —NHS(O)₂R^{Z}, —NR^{Z}₂, —NHR^{Z} and —OR^{Z}.

For example, each Y group may independently represent a group selected from the list consisting of: cyano, halogen, N₃, —C(O)R^{Z}, —C(O)OR^{Z}, —OC(O)R^{Z}, —C(O)NHR^{Z}, —NHC(O)R^{Z}, —NHC(O)OR^{Z}, —OC(O)NHR^{Z}, —OP(O)₂OR^{Z}, —S(O)₂NHR^{Z}, —NHS(O)₂R^{Z}, and —OR^{Z}. Each Y group may independently represent a group selected from the list consisting of: cyano, halogen, N₃, —C(O)R^{Z}, —C(O)OR^{Z}, —OC(O)R^{Z}, —C(O)NHR^{Z}, —NHC(O)R^{Z}, —NHC(O)OR^{Z}, —OC(O)NHR^{Z} and —OR^{Z}. Each Y group may independently represent a group selected from the list consisting of: cyano, halogen, —C(O)R^{Z}, —C(O)OR^{Z}, —OC(O)R^{Z}, —C(O)NHR^{Z}, —NHC(O)R^{Z}, and —OR^{Z}.

In one embodiment, the compound of formula (I) or formula (II) includes 5 or fewer Y groups, such as 4 or fewer. Preferably the compound of formula (I) or formula (II) includes 3 or fewer Y groups, such as 2 or fewer Y groups, or more preferably 1 Y group. The compound of formula (I) or formula (II) may include from 1 to 5 Y groups, such as from 1 to 3 Y groups. Most preferably the compound of formula (I) or formula (II) includes no Y groups.

The compound of formula (I) or formula (II) may include 5 or fewer Y groups each independently selected from the list consisting of: cyano, halogen, N₃, —C(O)R^{Z}, —C(O)OR^{Z}, —OC(O)R^{Z}, —C(O)NHR^{Z}, —NHC(O)R^{Z}, —NHC(O)OR^{Z}, —OC(O)NHR^{Z}, —OP(O)₂OR^{Z}, —S(O)₂NHR^{Z}, —NHS(O)₂R^{Z}, and —OR^{Z}. For example, the compound of formula (I) or formula (II) may include 3 or fewer Y groups each independently selected from the list consisting of: cyano, halogen, N₃, —C(O)R^{Z}, —C(O)OR^{Z}, —OC(O)R^{Z}, —C(O)NHR^{Z}, —NHC(O)R^{Z}, —NHC(O)OR^{Z}, —OC(O)NHR^{Z} and —OR^{Z}.

For instance, the compound of formula (I) or formula (II) may include 3 or fewer Y groups selected from the list consisting of: cyano, halogen, —C(O)R^{Z}, —C(O)OR^{Z}, —OC(O)R^{Z}, —C(O)NHR^{Z}, —NHC(O)R^{Z}, and —OR^{Z}.

Each R^{Z} independently represents C1-10 alkyl, C2-10 alkenyl, C6-10 aryl, or C4-10 heterocyclyl. For example, each R^{Z} may independently represent C1-6 alkyl, C2-6 alkenyl, C6 aryl, or C4-6 heterocyclyl. Each R^{Z} may independently represent C1-10 alkyl or C2-10 alkenyl, such as C1-6 alkyl or C2-6 alkenyl, or C1-4 alkyl or C2-4 alkenyl.

### Preferred Embodiments

In one embodiment the compound of Formula (I) is of Formula (Ia):

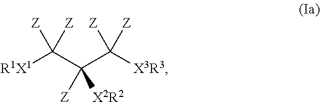

wherein the groups may take any of the definitions above.

In one embodiment the compound of Formula (I) is of Formula (Ib):

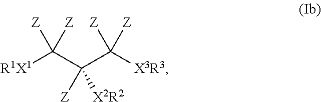

wherein the groups may take any of the definitions above.

Preferably each Z group represents H. Preferably R¹ represents a carbohydrate group, for example a sugar group, more preferably a monosaccharide group or disaccharide group. Preferably X¹ and the X group bonded to the hydrocarbon group represents a linker group selected from the list consisting of: ether, thioether, amine, phosphine, ester, thioester, amide, urea, thiourea, carbamate, phosphate and sulfonamide. Preferably the X group bonded to H represents a group selected from the list consisting of: —O—, —S—, —NH—, —PH—, —C(O)O—, —C(O)NH—, —OP(O)₂O—, and —S(O)₂NH—. Preferably n is 1.

Preferably Formula (I) represents Formula (Ic):

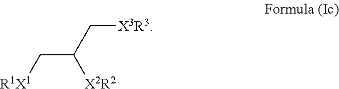

Thus, preferably R¹ represents a carbohydrate group; n is 1; either: a) R² represents H and R³ represents the hydrocarbon group, or b) R² represents the hydrocarbon group and R³ represents H; the hydrocarbon group represents C6-C30 alkyl or C6-C30 alkenyl, optionally including one or more Y group; each Y group independently represents a group selected from the list consisting of: cyano, halogen, N₃, —C(O)R^{Z}, —C(O)OR^{Z}, —OC(O)R^{Z}, —C(O)NHR^{Z}, —NHC(O)R^{Z}, —NHC(O)NHR^{Z}, —NHC(O)OR^{Z}, —OC(O)NHR^{Z}, —OP(O)₂OR^{Z}, —S(O)₂NHR^{Z}, —NHS(O)₂R^{Z}, —NR^{Z}₂, —NHR^{Z} and —OR^{Z}; each R^{Z} independently represents C1-10 alkyl, C2-10 alkenyl, C6-10 aryl, or C4-10 heterocyclyl; X¹ and the X group bonded to the hydrocarbon group represents a linker group selected from the list consisting of: ether, thioether, amine, phosphine, ester, thioester, amide, urea, thiourea, carbamate, phosphate and sulfonamide; and the X group bonded to H represents a group selected from the list consisting of: —O—, —S—, —NH—, —PH—, —C(O)O—, —C(O)NH—, —OP(O)₂O—, and —S(O)₂NH—.

More preferably R¹ represents a monosaccharide or disaccharide group (yet more preferably R¹ represents a disaccharide group); n is 1; either: a) R² represents H and R³ represents the hydrocarbon group, or b) R² represents the hydrocarbon group and R³ represents H; the hydrocarbon group represents C10-C24 alkyl or C10-C24 alkenyl, optionally including one or more Y group; each Y group independently represents a group selected from the list consisting of: halogen, —C(O)OR^{Z}, —OC(O)R^{Z}, —C(O)NHR^{Z}, —NHC(O)R^{Z}, —NHC(O)NHR^{Z}, —NHC(O)OR^{Z}, —OC(O)NHR^{Z}, and —OR^{Z}; each R^{Z} independently represents C1-10 alkyl or C2-10 alkenyl; X¹ and the X group bonded to the hydrocarbon group represents a linker group selected from the list consisting of: ether, ester, amide, urea, and carbamate; and the X group bonded to H represents a group selected from the list consisting of: —O—, —S—, —C(O)O—, and —C(O)NH—.

Yet more preferably R¹ represents a disaccharide group; n is 1; R² represents H; R³ represents the hydrocarbon group; the hydrocarbon group represents C10-C24 alkyl or C10-C24 alkenyl; X¹ and the X group bonded to the hydrocarbon group represents a linker group selected from the list consisting of: ether, ester, amide, urea, carbamate; and the X group bonded to H represents a group selected from the list consisting of: —O—, —S—, —C(O)O— and —C(O)NH—.

In one embodiment the compound of Formula (I) is of Formula (Id):

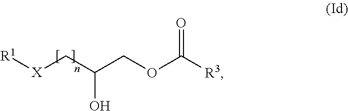

wherein the groups R¹, R², R³, may take any of the definitions above, and X is either absent or is —O—, —NR^{a}—, —S— or —CR^{a}R^{b}—, wherein R^{a} and R^{b} are independently selected from the group consisting of hydrogen and C1-C4 alkyl, and n is an integer of from 1 to 6, e.g. 1, 2 or 3.

For example, the compound of Formula (I) may be of Formula (Je):

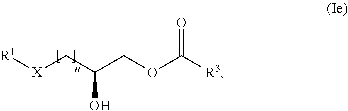

wherein the groups R¹, R², R³, may take any of the definitions above, and X is either absent or is —O—, —NR^{a}—, —S— or —CR^{a}R^{b}—, wherein R^{a} and R^{b} are independently selected from the group consisting of hydrogen and C1-C4 alkyl, and n is an integer of from 1 to 6, e.g. 1, 2 or 3.

In one embodiment:
R¹ is a carbohydrate group or derivative thereof;
X is either absent or is —O—, —NR^{a}—, —S— or —CR^{a}R^{b}—, wherein R^{a} and R^{b} are independently selected from the group consisting of hydrogen and C1-C4 alkyl;
n is an integer of from 1 to 6, e.g. 1, 2 or 3;
the hydrocarbon group is a C10-C24 alkyl, alkenyl or alkynyl group or a derivative thereof.

In one embodiment:
the disaccharide group is represented by the formula:

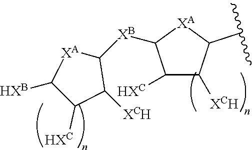

wherein:
   each X^{A} group independently represents —CH₂—, —O—, —S—, —NH— or —PH—;
   each X^{B} group independently represents —O—, —S—, —NH—, —PH—, or —(CH2)ₘX^{C}—.
   each X^{C} group independently represents —O—, —S—, —NH—, or —PH—;
   each n represents 1 or 2;
   each m represents 1 or 2; and
   the disaccharide group is optionally substituted with one or more Y group, and
the hydrocarbon group represents C10-C26 alkyl or C10-C26 alkenyl, optionally substituted with 3 or fewer Y groups, and
X¹ is selected from the list consisting of: ether, thioether, ester, thioester, amide, urea, thiourea and carbamate, and
the X² or X³ group bonded to the hydrocarbon group is selected from the list consisting of: ether, amine, ester, thioester, amide, urea, thiourea, carbamate, phosphate and sulfonamide, and
the X² or X³ group bonded to H is selected from the list consisting of: —O—, —S—, —NH—
Y is selected from the list consisting of: cyano, halogen, N₃, —C(O)R^{Z}, —C(O)OR^{Z}, —OC(O)R^{Z}, —C(O)NHR^{Z}, —NHC(O)R^{Z}, —NHC(O)OR^{Z}, —OC(O)NHR^{Z}, —OP(O)₂OR^{Z}, —S(O)₂NHR^{Z}, —NHS(O)₂R^{Z}, and —OR^{Z}, wherein R^{Z} represents C1-10 alkyl or C2-10 alkenyl.

### Preferably:

the disaccharide group is represented by the formula:

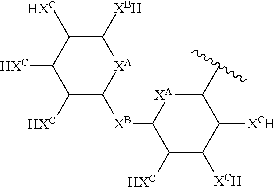

wherein:
   each X^{A} group independently represents —O— or —S—;
   and each X^{B} group independently represents —O—, —S—, —(CH₂)ₘO—, or —(CH₂)ₘS—,
   each X^{C} group independently represents —O— or —S—
   each m represents 1 or 2; and
   the disaccharide group is optionally substituted with from 0 to 2 Y groups, and
the hydrocarbon group represents C14-C22 alkyl or C14-C22 alkenyl, optionally substituted with 1 Y group, and
X¹ is selected from the list consisting of: ether and thioether, and
the X² or X³ group bonded to the hydrocarbon group is selected from the list consisting of: ester and amide, and
the X² or X³ group bonded to H is selected from the list consisting of: —O— and —S—
Y is selected from the list consisting of: cyano, halogen, —C(O)R^{Z}, —C(O)OR^{Z}, —OC(O)R^{Z}, —C(O)NHR^{Z}, —NHC(O)R^{Z}, and —OR^{Z}, wherein R^{Z} represents C1-6 alkyl or C2-6 alkenyl.
   More preferably:
The disaccharide group is a digalactosyl group,
the hydrocarbon group represents C14-C22 alkenyl, wherein the alkenyl group contains from 1 to 4 alkene groups that is/are not terminal alkene group(s),
X¹ represents an ether,
the X² or X³ group bonded to the hydrocarbon group represents an ester, and
the X² or X³ group bonded to H represents —O—.

### Applications

The compound of the first aspect, and a composition thereof as defined by the third aspect, is for use as a medicament. According to the second aspect, the compound of Formula (I), and according to the fourth aspect a composition thereof, is for use as a chemotherapeutic agent, an inhibitor of protein translation, a cell sensitising agent, an antiproliferative agent, an antiviral agent or an adjuvant, or for use in the treatment or prevention of disease or condition selected from the group consisting of: viral infection, cancer, and CNS-related disorders.

The present disclosure also provides a compound of Formula (I), or a composition thereof, for use in the treatment or prevention of a disease or condition which is caused by dysregulation of protein translation. The present disclosure also provides a method of treating a disease or disorder, such as viral infection, cancer, or CNS-related disorder, in a subject in need thereof, comprising administering to the subject a therapeutically effective amount of a compound of Formula (I), or a composition thereof. These are examples of diseases and conditions caused by dysregulation of protein translation. The disease or condition may be selected from the group comprising cancer, Alzheimer's disease and autism spectrum disorder. The disease or condition may be cancer.

Thus, the present disclosure provides a method of treating a disease or disorder, such as cancer, viral infection, or CNS-related disorders, in a subject in need thereof, comprising administering to the subject a therapeutically effective amount of a compound of Formula (I), or a composition thereof, or a pharmaceutically acceptable salt thereof.

The present disclosure also provides a kit comprising:
as a first therapeutic agent, a compound of Formula (I), or a composition thereof, and
as a second therapeutic agent, an anti-cancer agent,
wherein the anti-cancer agent is provided in a form suitable for, and/or with instructions for, administration in a daily dosage which is significantly reduced (e.g. by 10% or more, or 20% or more, or 30% or more) compared to the dosage of the anti-cancer agent if administered alone. The first and second therapeutic agents may be intended to be administered simultaneously, sequentially or separately. The anti-cancer agent may be a chemotherapeutic agent.

The invention may provide in another aspect a compound of Formula (I) or a pharmaceutically acceptable salt thereof, for use as an inhibitor of protein translation. More preferably a compound of the invention may inhibit protein translation by inhibiting eukaryotic ribosome activity, in particular, ribosome recruitment. The compound may selectively inhibit eIF4A dependent or independent translation. A compound of the invention may inhibit protein translation by selectively reducing translation of mRNAs with long structured UTRs. Thus, the present disclosure provides a method of inhibiting protein translation comprising administering a composition, compound or pharmaceutically acceptable salt thereof according to the invention to a cell or a subject.

The disease or condition treated by the compound of Formula (I), and compositions thereof, may be selected from the group consisting of: viral infections, cancer, Alzheimer's disease and autism spectrum disorder. Preferably the compound of Formula (I) is for use in the treatment or prevention of viral infections.

Preferably the disease or condition is selected from the list consisting of: viral infection, cancer, and CNS-related disorders (e.g. Alzheimer's disease, Parkinson's disease, Huntingdon's disease, frontotemporal dementia, motor neurone disease, muscle wasting and autism spectrum disorder). The disease or condition may be selected from the list consisting of: viral infection, cancer, Alzheimer's disease, Parkinson's disease, Huntingdon's disease, muscle wasting and autism spectrum disorder.

In relation to antiviral compounds and the treatment or prevention of viral infections, the virus may be an enterovirus, and/or selected from the group consisting of: a virus of the order Herpesvirales, such as a herpes virus (e.g. herpes simplex virus (HSV), Abalone herpesvirus (dsDNA) and human cytomegalovirus (HCMV)); Viral haemorrhagic septicaemia virus (VHSV); Infectious haematopoietic necrosis virus (IHNV); human immunodeficiency virus (HIV); respiratory viruses (e.g. influenza (e.g. type A, B, C, and D, especially type A, and more especially influenza viruses H1N1, H2N3, and LPAI H2N3); coronaviruses (e.g. human coronaviruses, including SARS-CoV, MERS-CoV, HCoV-229E, and SARS-CoV-2); rhinoviruses (e.g. human rhinovirus (HRV)); foot-and-mouth disease virus (FMDV); human cytomegalovirus (HCMV); noroviruses; ebolaviruses; polioviruses (e.g. type 1, 2 and 3, especially type 1); Chikungunya virus (CHIKV); a virus of the genus Flavivirus (e.g. Zika virus), Hepatoviruses (e.g. type A, B, C, D and E, especially types C and E); Lassa virus; encephalomyocarditis virus (EMCV); Crimean-Congo haemorrhagic fever virus; a virus of the genus Orthoreovirus (e.g. Piscine orthoreovirus (dsRNA)); a virus of the family Orthomyxoviridae (e.g. Infectious salmon anaemia virus); Tilapia lake virus (RNA−); a virus of the genus alphanodavirus, e.g. Covert mortality nodavirus (RNA+); a virus of the family Iridoviridae (e.g. Shrimp hemocyte iridescent virus (dsDNA)); a virus of the genus orthobunyavirus (e.g. Schmallenberg orthobunyavirus) and combinations thereof. The virus may be an RNA virus (e.g. RNA+ and/or RNA−) and/or a DNA virus. Preferably the virus is an RNA virus, for example an RNA virus selected from the list of viruses above. Preferably the virus is an influenza virus, for example influenza type A, e.g. H1N1 or H2N3 (e.g. LPAI H2N3), or a virus of the genus Flavivirus (preferably Zika virus), or a virus of the genus orthobunyavirus (preferably Schmallenberg orthobunyavirus), or a coronavirus (e.g. human coronaviruses, including SARS-CoV, MERS-CoV, HCoV-229E, and SARS-CoV-2). Preferably the virus is an influenza virus, for example influenza type A, e.g. H1N1 or H2N3 (e.g. LPAI H2N3), or a virus of the genus Flavivirus (preferably Zika virus), or a virus of the genus orthobunyavirus (preferably Schmallenberg orthobunyavirus).

The invention may further provide a product containing at least (i) a compound of Formula (I), a composition thereof, or a pharmaceutically acceptable salt thereof, (ii) and a chemotherapeutic agent, as a combined preparation for simultaneous, separate or sequential use in an anticancer therapy. The compound, composition or salt thereof and the chemotherapeutic agent may be provided in the same or different preparations.

The cancer may be selected from carcinoma, lymphoma, blastoma, sarcoma, and leukemia. More particularly, examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, renal cancer, vulval cancer, thyroid cancer, hepatic carcinoma, gastric cancer, melanoma, and various types of head and neck cancer. The cancer may be selected from breast, lung or ovarian cancer.

The compound of the present invention (or composition thereof or pharmaceutically acceptable salt thereof) may be used to sensitise cells, such as cancer cells, to chemotherapeutic agents, for example to cis-diamminedichloroplatinum(II) (Cisplatin™) or (7S,9S)-7-[(2R,4S,5S,6S)-4-amino-5-hydroxy-6-methyloxan-2-yl]oxy-6,9,11-trihydroxy-9-(2-hydroxyacetyl)-4-methoxy-8,10-dihydro-7H-tetracene-5,12-dione (Doxorubicin™), or to any other chemotherapeutic agent.

A cell sensitising agent may act to sensitise cells to subsequent or simultaneous treatment or prevention with another active agent. For example, a cell sensitising cell may act to sensitise cell to an anti-cancer agent, such as a chemotherapeutic agent, such that the anti-cancer agent is more efficacious or is efficacious at lower doses. The compound or composition thereof or pharmaceutical salts thereof of the present invention may have an additive therapeutic effect when administered in combination with an anti-cancer agent, such as a chemotherapeutic agent.

In another aspect the invention provides a compound of Formula (I), or a composition thereof, or a pharmaceutically acceptable salt thereof for use in the treatment or prevention of a disease or condition, wherein the compound, composition or pharmaceutically acceptable salt thereof is administered as a first therapeutic agent, and a further therapeutic agent is administered as a second therapeutic agent wherein the dosage, preferably the daily dosage, of the second therapeutic agent is significantly reduced (e.g. by 10% or more, or 20% or more, or 30% or more) compared to the daily dosage of the second therapeutic agent when administered alone. Preferably the first and second therapeutic agents are for the treatment or prevention of cancer; the first therapeutic agent may be a cell sensitising agent which sensitises cells to the action of the second therapeutic agent. Preferably the second therapeutic agent is an anti-cancer agent, preferably a chemotherapeutic agent. The first and second therapeutic agents may be administered simultaneously, sequentially or separately. The amount of anti-cancer drug, in particular chemotherapeutic agent, needed to be efficacious against a particular cancer may be reduced between about 5 and about 100 fold by administering the compound, composition or pharmaceutically acceptable salt thereof of the invention. The daily dose of the chemotherapeutic agent may be reduced by about 5 to about 100 fold, preferably at least about 5 fold, more preferably about 5 to about 50 fold, or about 5 to about 40 fold, or about 20 to about 50 fold, or about 20 to about 40 fold, or about 40 fold.

The present disclosure provides a method of reducing the dosage required of an anti-cancer agent, the method comprising administering to a subject with cancer or to cancer cells an amount of a compound of Formula (I), or a composition thereof, or a pharmaceutically acceptable salt thereof, effective to sensitise the cancer cells to the anticancer agent. The anticancer agent may be a chemotherapeutic agent. The present disclosure provides a method of enhancing the therapeutic activity of an anti-cancer agent which comprises administering to a patient an amount of a compound of Formula (I), or a composition thereof, or a pharmaceutically acceptable salt thereof, effective to sensitise cancer cells in the patient to the anti-cancer agent. The compound, composition or salt may be administered simultaneously, sequentially or separately to the anti-cancer agent. The compound, composition or salt may be used to sensitise cells, such as cancer cells, to known chemotherapeutic agents, for example to cis-diamminedichloroplatinum(II) (Cisplatin™) or (7S,9S)-7-[(2R,4S,5S,6S)-4-amino-5-hydroxy-6-methyloxan-2-yl]oxy-6,9,11-trihydroxy-9-(2-hydroxyacetyl)-4-methoxy-8,10-dihydro-7H-tetracene-5,12-dione (Doxorubicin™), or to any other chemotherapeutic agent.

A chemotherapeutic agent is a chemical compound useful in the treatment or prevention of cancer. Examples of chemotherapeutic agents include chemical compounds useful in the treatment or prevention of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e. g., calicheamicin, especially calicheamicin gammall and calicheamicin omegall (see, e.g., Agnew, Chem Intl. Ed. Engl, 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAM YCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizoffran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Illinois), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluorometlhylornithine (DMFO); retinoids such as retinoic acid; capecitabine; combretastatin; leucovorin (LV); oxaliplatin, including the oxaliplatin treatment regimen (FOLFOX); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above. Also included in this definition are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY1 17018, onapristone, and FARESTON-toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, RaIf and H-Ras; ribozymes such as a VEGF expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above. In one embodiment, the second therapeutic agent is cis-diamminedichloroplatinum(II) (Cisplatin™) or (7S,9S)-7-[(2R,4S,5S,6S)-4-amino-5-hydroxy-6-methyloxan-2-yl]oxy-6,9,11-trihydroxy-9-(2-hydroxyacetyl)-4-methoxy-8,10-dihydro-7H-tetracene-5,12-dione (Doxorubicin™), or any other chemotherapeutic agent.

The therapeutic dose of a compound of Formula (I), or a composition thereof, may vary depending on the type and severity of the disease or condition to be treated. The compound of Formula (I), or a composition thereof, may be provided in, or administered in, a dose of from 3 mg to 1.2 g, for example from 3 mg to 1 g, or from 3 mg to 500 mg, such as from 3 mg to 300 mg. These amounts may be administered daily. The compound of Formula (I), or a composition thereof, may be administered in a single dose, or in multiple doses. The multiple doses may be administered over the course of one day or over several days, for example over 2 or 3 days, or over 4 or 5 days or more. The dose per day may be between about 60 and 300 mg per 70 kg of subject weight per day. These values are particularly applicable to the compound of Formula (I), or the composition thereof, being used in the treatment or prevention of cancer, for example as a chemotherapeutic agent.

For the treatment or prevention of CNS-related disorders such as Alzheimer's disease, Parkinson's disease, Huntingdon's disease, frontotemporal dementia, motor neurone disease, muscle wasting or autism spectrum disorder, or viral infection, the dose of a compound of Formula (I), or a composition thereof, used may be between about 3 mg and about 120 mg per day. The compound of Formula (I), or a composition thereof, may be administered in a single dose, or in multiple doses. The multiple doses may be administered over the course of one day or over several days, for example over 2 or 3 days, or over 4 or 5 days or more. The dose per day may be between about 6 and 30 mg per 70 kg of subject weight per day.

For the treatment or prevention of cancer, wherein the compound of Formula (I), or a composition thereof, is being used to sensitise cancer cells to a different chemotherapeutic agent the dose per day may be between about 10 and 70 mg per 70 kg of subject weight per day. This may allow the dose of chemotherapeutic agent to be reduced by at least about 5 fold compared the dose of chemotherapeutic agent recommended in the absence of a compound of Formula (I), or a composition thereof. The daily dose of a chemotherapeutic agent may be reduced by about 5 to about 100 fold, preferably at least about 5 fold, more preferably about 5 to about 50 fold, or about 5 to about 40 fold, or about 20 to about 50 fold, or about 20 to about 40 fold, or about 40 fold.

Preferably compounds of Formula (I), and compositions thereof, do not have any significant side effects when administered to a subject. Preferably at the doses required for efficacy the compounds are not toxic to a subject.

The compound of Formula (I) may be formulated as a prodrug or a protected formula. The compound may be a prodrug or a protected form of the compound which releases the compound after administration to a subject. For example, the compound may carry a protective group which is split off by hydrolysis in body fluids, e.g., in the bloodstream, thus releasing the active compound or is oxidized or reduced in body fluids to release the compound. Reference to a “prodrug” is intended to indicate a compound that may be converted under physiological conditions or by solvolysis to a biologically active compound of the invention. Thus, the term “prodrug” refers to a metabolic precursor of a compound of the invention that is pharmaceutically acceptable. A prodrug may be inactive when administered to a subject in need thereof, but is converted in vivo to an active compound of the invention. Prodrugs are typically rapidly transformed in vivo to yield the parent compound of the invention, for example, by hydrolysis in blood. The prodrug compound often offers advantages of solubility, tissue compatibility or delayed release in a subject.

The term “prodrug” may include any covalently bonded carriers which release the active compound of the invention in vivo when such prodrug is administered to a subject. Prodrugs of a compound of Formula (I) may be prepared by modifying functional groups present in Formula (I) in such a way that the modifications are cleaved, either in routine manipulation or in vivo, to the parent compound of Formula (I). Prodrugs include compounds of Formula (I) wherein a hydroxy, amino or mercapto group is bonded to any group that, when the prodrug of the compound of the invention is administered to a mammalian subject, cleaves to form a free hydroxy, free amino or free mercapto group, respectively. Examples of prodrugs include, but are not limited to, acetate, formate and benzoate derivatives of alcohol and acetamide, formamide, and benzamide derivatives of amine functional groups in the compounds of the invention and the like.

A discussion of prodrugs may be found in “Smith and Williams' Introduction to the Principles of Drug Design,” H. J. Smith, Wright, Second Edition, London (1988), which is incorporated in full by reference herein.

The invention provides a pharmaceutical composition comprising: a compound of formula (I), or a pharmaceutically acceptable salt thereof or a pharmaceutically acceptable solvate thereof; and a pharmaceutically acceptable carrier, diluent or excipient. The carrier may, for example, be water or an aqueous fluid such as saline. However, the skilled person will be well aware of carriers, diluents or excipients that are pharmaceutically acceptable.

The pharmaceutical composition may also comprise one or more further anti-cancer agent, such as a chemotherapeutic agent. The anti-cancer agent, such as a chemotherapeutic agent, may comprise cis-diamminedichloroplatinum(II) (Cisplatin™) or (7S,9S)-7-[(2R,4S,5S,6S)-4-amino-5-hydroxy-6-methyloxan-2-yl]oxy-6,9,11-trihydroxy-9-(2-hydroxyacetyl)-4-methoxy-8,10-dihydro-7H-tetracene-5,12-dione (Doxorubicin™), or any other chemotherapeutic agent.

The invention further provides a nutraceutical composition comprising a compound of formula (I), or a pharmaceutically acceptable salt thereof or a pharmaceutically acceptable solvate thereof. The nutraceutical composition may be an animal feed. The nutraceutical composition may be an animal feed.

It is common for animal feed to be provided as a composition including other components, for example as a mixture. The animal feed may therefore also include one or more vitamins, minerals, chemical preservatives, antibiotics, fermentation products (e.g. spent yeast), molasses, grain (e.g. spent grain, and/or maize, soybean, wheat, oats, barley, and/or rice), seaweed, fodder (e.g. hay, straw, silage, compressed and pelleted feeds, oils and mixed rations, and sprouted grains and legumes), peanut shell, bean pods (e.g. soy bean pods), corn bract and/or corn cobs. Preferably the animal feed comprises fermentation products, molasses, grain, seaweed, fodder, peanut shell, bean pods, corn bract and/or corn cobs.

The animal feed may include these components in an amount of 30 wt % or more, such as 50 wt % or more, or 70 wt % or more, such as 90 wt % or more, or 95 wt % or more, such as 98 wt % or more. The amount may be 99.9 wt % or less, such as 99.5 wt % or less, or 99 wt % or less, for example 98.5 wt % or less, or 98 wt % or less, for example 95 wt % or less. The amount of these components in the animal feed may be from 30 to 99.9 wt %, such as from 50 to 99.5 wt %, or from 70 to 99 wt %.

For example, the animal feed may comprise fermentation products, molasses, grain, seaweed, fodder, peanut shell, bean pods, corn bract and/or corn cobs in an amount of 30 wt % or more, such as from 50 to 99.5 wt %.

The nutraceutical composition (e.g. animal feed) may include the compound of formula (I) in what might seem like quite low concentrations. However, due to the amount of feed that animals can eat, the overall amount of the compound eaten can be sufficient to provide a technical effect. Therefore, the nutraceutical composition (e.g. animal feed) may include the compound of formula (I) in an amount of 0.000001 wt % or more, such as 0.000005 wt % or more, or 0.00001 wt % or more, preferably 0.00005 wt % or more, or 0.0001 wt % or more, for example 0.0002 wt % or more, or 0.0003 wt % or more, or 0.0004 wt % or more. The amount of the compound of formula (I) in the nutraceutical composition (e.g. animal feed) may be 10 wt % or less, such as 5 wt % or less, or 1 wt % or less, such as 0.5 wt % or less, or 0.1 wt % or less, such as 0.05 wt % or less, such as 0.01 wt % or less, preferably 0.005 wt % or less, or 0.001 wt % or less, for example 0.0008 wt % or less, or 0.0007 wt % or less, or 0.0006 wt % or less. The amount of the compound in the nutraceutical composition may be from 0.000001 wt % to 10 wt %, for example from 0.00001 wt % to 1 wt %, or from 0.00005 wt % to 0.005 wt %, preferably from 0.0001 wt % to 0.001 wt %, or from 0.0003 wt % to 0.0008 wt %.

The compound in the nutraceutical composition (e.g. animal feed) may have been extracted from fruit (e.g. tomatoes) that contain the compound. Preferably the compound is added to the nutraceutical composition in fruit (e.g. dehydrated fruit) that contains the compound. Therefore, the composition preferably contains fruit (e.g. tomatoes) that contains the compound. The composition may contain the fruit (e.g. tomatoes) in a dry (i.e. dehydrated) amount of 0.0001 wt % or more, such as 0.001 wt % or more, or 0.005 wt % or more, such as 0.01 wt % or more, preferably 0.02 wt % or more, or 0.05 wt % or more, or 0.08 wt % or more, such as 0.09 wt % or more, or 0.1 wt % or more, or 0.15 wt % or more. The dry amount of fruit (e.g. tomatoes) in the composition may be 20 wt % or less, such as 10 wt % or less, or 5 wt % or less, preferably 2 wt % or less, for example 1 wt % or less, or 0.8 wt % or less, such as 0.6 wt % or less, or 0.4 wt % or less, or 0.3 wt % or less. The dry amount of the fruit (e.g. tomatoes) in the composition may be from 0.0001 wt % to 20 wt %, such as from 0.005 wt % to 5 wt %, preferably from 0.02 wt % to 2 wt %, such as from 0.08 wt % to 1 wt %.

The feed may either contain the compound of Formula (I) in an amount of from 0.000001 wt % or more (e.g. from 0.000001 wt % to 10 wt %, or from 0.0001 wt % to 0.001 wt %), or contain fruit (e.g. tomatoes) in a dry amount of from 0.0001 wt % or more (e.g. from 0.0001 wt % to 20 wt %, or from 0.02 wt % to 2 wt %), and contain other components that are vitamins, minerals, chemical preservatives, antibiotics, fermentation products (e.g. spent yeast), molasses, grain (e.g. spent grain, and/or maize, soybean, wheat, oats, barley, and/or rice), seaweed, fodder (e.g. hay, straw, silage, compressed and pelleted feeds, oils and mixed rations, and sprouted grains and legumes), peanut shell, bean pods (e.g. soy bean pods), corn bract and/or corn cobs, where the other components are included in an amount of 30 wt % or more (e.g. from 30 to 99.9 wt %). The skilled person will appreciate that any remainder can made up with other components, such that the total composition adds up to 100%.

The fruit may be whole or processed. The fruit may be partially or totally dehydrated, or may not be dehydrated.

The animals being fed the animal feed may include livestock. The livestock may be ruminants or non-ruminants. The livestock may be cattle, sheep, goats, pigs, horses, donkeys, zebu, bali cattle, yak, water buffalo, gayal, reindeer, camel (e.g. Bactrian camel, Arabian camel), llama, alpaca, poultry (e.g. chicken), rabbit, and/or guinea pig. The animals may include pets (companion animals), such as dogs, cats, rabbits, ferrets, pigs, rodents (e.g. gerbils, hamsters, chinchillas, rats, mice, and guinea pigs), birds (e.g. parrots, passerines, and fowls), reptiles (e.g. turtles, lizards, snakes, and iguanas), equine (e.g. horses, ponies and donkeys) aquatic pets (e.g. fish, freshwater snails, and saltwater snails), amphibians (e.g. frogs and salamanders), and/or arthropods (e.g. tarantulas and hermit crabs). The animals may be for meat-based food products, non-meat-based food products (e.g. eggs, milk), and/or non-food products such as wool and leather.

It will be understood that the above parameters may specifically be used to define the eighth and/or ninth aspects.

The compound of Formula (I), or the pharmaceutically acceptable salt thereof, may be according to any of the definitions given above. The nutraceutical composition may comprise: a compound of Formula (I), or a pharmaceutically acceptable salt thereof; and a nutraceutically acceptable carrier, diluent or excipient. The carrier may, for example, be water or an aqueous fluid such as saline or a sugar solution. However, the skilled person will be well aware of carriers, diluents or excipients that are nutraceutically acceptable.

Compounds for use according to the invention can be provided alone or in combination with other compounds, for example they may be provided in the presence of a liposome, an adjuvant, or any pharmaceutically acceptable carrier, diluent or excipient, in a form suitable for administration to a subject such as a mammal, for example, humans, cattle, sheep, etc. If desired, treatment or prevention with a compound according to the invention may be combined with more traditional and existing therapies for the therapeutic indications described herein. For example, in the treatment or prevention of cancer compositions according to the invention may be administered in combination with one or more additional anti-cancer therapies. Examples of anti-cancer therapies include surgery, radiation therapy (radiotherapy), biotherapy, immunotherapy, chemotherapy, or a combination of these therapies. Chemotherapy may include the administration of one or more chemotherapeutic agents. The compound, or composition thereof, according to the invention and the one or more additional anti-cancer therapies, such as one or more chemotherapeutic agents, may be administered separately, sequentially or simultaneously.

The combined administration of a compound of Formula (I) and an additional anti-cancer therapy includes coadministration or concurrent administration, using separate formulations or a single pharmaceutical formulation, and consecutive administration in either order, wherein optionally there is a time period while both (or all) active agents simultaneously exert their biological activities.

“Pharmaceutically acceptable carrier, diluent or excipient” includes any adjuvant, carrier, excipient, glidant, sweetening agent, diluent, preservative, dye/colorant, flavour enhancer, surfactant, wetting agent, dispersing agent, suspending agent, stabilizer, isotonic agent, solvent, or emulsifier that has been approved, for example, by the United States Food and Drug Administration or other governmental agency as being acceptable for use in humans or domestic animals.

The compounds of the present invention may be administered in the form of pharmaceutically acceptable salts. In such cases, pharmaceutical compositions in accordance with this invention may comprise a salt of such a compound, preferably a physiologically acceptable salt, which are known in the art. In some embodiments, the term “pharmaceutically acceptable salt” as used herein means an active ingredient comprising compounds of Formula (I) used in the form of a salt thereof, particularly where the salt form confers on the active ingredient improved pharmacokinetic properties as compared to the free form of the active ingredient or other previously disclosed salt form. Pharmaceutically acceptable salts of the compounds of the present invention may be used to modify solubility or hydrolysis characteristics, or to produce a sustained release formulations.

The term “pharmaceutically acceptable salt” encompasses all acceptable salts, including: acetate, lactobionate, benzenesulfonate, laurate, benzoate, malate, bicarbonate, maleate, bisulfate, mandelate, bitartarate, mesylate, borate, methylbromide, bromide, methylnitrite, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutame, stearate, glycollylarsanilate, sulfate, hexylresorcinate, subacetate, hydradamine, succinate, hydrobromide, tannate, hydrochloride, tartrate, hydroxynaphthoate, teoclate, iodide, tosylate, isothionate, triethiodide, lactate, panoate and valerate and the like. Pharmaceutically acceptable salts of the compounds of this invention may also include those formed from cations such as sodium, potassium, aluminium, calcium, lithium, magnesium, zinc, and from bases such as ammonia, ethylenediamine, N-methyl-glutamine, lysine, arginine, ornithine, choline, N,N′-dibenzylethylene-diamine, chloroprocaine, diethanolamine, procaine, N-benzylphenethyl-amine, diethylamine, piperazine, tris(hydroxymethyl)aminomethane, and tetramethylammonium hydroxide. The pharmaceutically acceptable salt may, for example, be one of those set out in P. H. Stahl and C. G. Wermuth, editors, Handbook of Pharmaceutical Salts: Properties, Selection and Use, Weinheim/Zürich:Wiley-VCH/VHCA, 2002.

Compositions containing a compound of Formula (I) may be pharmaceutical compositions and/or pharmaceutical formulations. Pharmaceutical formulations of the compound of formula (I), and compositions thereof, will typically include one or more carriers acceptable for the mode of administration of the preparation, be it by injection, inhalation, topical administration, lavage, enteral or other modes suitable for the selected treatment or prevention. Suitable carriers are those known in the art for use in such modes of administration.

Suitable pharmaceutical compositions may be formulated by means known in the art and their mode of administration and dose determined by the skilled practitioner. For parenteral administration, a compound may be dissolved in sterile water or saline or a pharmaceutically acceptable vehicle used for administration of non-water soluble compounds such as those used for vitamin K. For enteral administration, the compound may be administered in a tablet, capsule or dissolved in liquid form. The table or capsule may be enteric coated, or in a formulation for sustained release. Many suitable formulations are known, including, polymeric or protein microparticles encapsulating a compound to be released, ointments, gels, hydrogels, or solutions which can be used topically or locally to administer a compound. A sustained release patch or implant may be employed to provide release over a prolonged period of time. Many techniques known to skilled practitioners are described in Remington: the Science & Practice of Pharmacy by Alfonso Gennaro, 20th ed., Williams & Wilkins, (2000). Formulations for parenteral administration may, for example, contain excipients, polyalkylene glycols such as polyethylene glycol, oils of vegetable origin, or hydrogenated naphthalenes. Biocompatible, biodegradable lactide polymer, lactide/glycolide copolymer, or polyoxyethylene-polyoxypropylene copolymers may be used to control the release of the compounds. Other potentially useful parenteral delivery systems for modulatory compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The compound of Formula (I), or composition thereof, may be administered by oral or non-oral, e.g., intramuscular, intraperitoneal, intravenous, intracisternal injection or infusion, subcutaneous injection, transdermal or transmucosal routes. In some embodiments, compounds or pharmaceutical compositions in accordance with this invention or for use in this invention may be administered by means of a medical device or appliance such as an implant, graft, prosthesis, stent, etc. Implants may be devised which are intended to contain and release such compounds or compositions. An example would be an implant made of a polymeric material adapted to release the compound over a period of time. The compound, or composition thereof, may be administered alone or as a mixture with a pharmaceutically acceptable carrier e.g., as solid formulations such as tablets, capsules, granules, powders, etc.; liquid formulations such as syrups, injections, etc.; injections, drops, suppositories, pessaries. In some embodiments, the compound, or composition thereof, may be administered by inhalation spray, nasal, vaginal, rectal, sublingual, or topical routes and may be formulated, alone or together, in suitable dosage unit formulations containing conventional non-toxic pharmaceutically acceptable carriers, adjuvants and vehicles appropriate for each route of administration.

The compound of Formula (I), or composition thereof, may be used to treat animals, including mice, rats, horses, cattle, sheep, dogs, cats, and monkeys. The compounds, and compositions thereof, may also be effective for use in humans. The term “subject” is intended to refer to an animal, preferably a mammal, preferably a human, who has been the object of treatment or prevention, observation or experiment. Accordingly, as used herein, a “subject” may be a human, non-human mammal, non-human primate, rat, mouse, cattle, horse, swine (e.g. pigs), sheep, goat, dog, cat, deer, camels, birds (e.g. poultry, such as chickens, and/or aquatic birds), etc.

As well as the treatment or prevention of conditions and/or diseases in humans, it is specifically envisaged that the present invention can be used to treat conditions and/or diseases, such as viral infections, in animals. Thus, the compounds, compositions thereof, methods and pharmaceutical compositions of Formula (I) may be used in the treatment or prevention of animals. Animals include non-human mammals, such as marine mammals (e.g. seals, whales and minks), non-human primates, monkeys, rats, mice, cattle, horses, swine, deer, camels, sheep, goats, dogs, and cats, fish (e.g. fin fish and/or shell fish) and birds (e.g. poultry and/or aquatic birds); viruses such as influenza are known to affect these animals. Preferably the animals are selected from the list consisting of: cattle, horses, swine, deer, sheep, goats and poultry. More preferably the animals are selected from the list consisting of: non-human primates, marine mammals (e.g. seals, whales and minks), cattle, horses, camels, swine, dogs, cats, fish (e.g. fin fish and/or shell fish) and birds (e.g. poultry and/or aquatic birds.

An “effective amount” of a compound according to the invention includes a therapeutically effective amount or a prophylactically effective amount. A “therapeutically effective amount” refers to an amount effective, at dosages and for periods of time necessary, to achieve the desired therapeutic result. A therapeutically effective amount of a compound may vary according to factors such as the disease state, age, sex, and weight of the individual, and the ability of the compound to elicit a desired response in the individual. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result. Typically, a prophylactic dose is used in subjects prior to or at an earlier stage of disease, so that a prophylactically effective amount may be less than a therapeutically effective amount. A suitable range for therapeutically or prophylactically effective amounts of a compound may be any integer from 0.1 ng/kg-1 g/kg, 0.1 ng/kg-0.5 g/kg, 0.05 ng/kg-150 μg/kg or 0.01 ng/kg-100 μg/kg.

The term ‘antiproliferative agents’ is intended to mean a pharmacological agent that blocks cellular, parasitic or viral growth. The term ‘adjuvant’ is intended to mean a pharmacological agent that would be added to, or administered with or alongside, a drug or therapeutic agent to enhance or aid the effect of the drug or therapeutic agent.

It will be appreciated that all preferred or optional features of the invention may be applied to all aspects of the invention, unless otherwise stated or incompatible.

The compounds of the present disclosure are not limited to any particular tautomer or stereoisomer. The compounds therefore include tautomeric or stereochemically isomeric forms thereof. However, the stereochemical configuration of the compounds of the invention may be as defined by the compound DGMG, as shown in FIG. 1. The references described herein are incorporated by reference.

The compounds of Formula (I), and compositions thereof, may be used as a feedstock for an animal or herd of animals, or as part of such feedstock.

It has been rationalised that the compounds of Formula (I), and compositions thereof, can be used to increase the yield of an animal or a herd of animals. The compounds of Formula (I), and compositions thereof, may be used to improve the appearance of the animal or herd thereof.

The use of the compounds of Formula (I) and compositions thereof may, therefore, be non-therapeutic. For example, the use of compounds of Formula (I) and compositions thereof may be solely cosmetic.

The appearance of the animal or herd thereof may be improved by increasing its level of alertness, level of movement, gait, reducing or removing excess discharge from mucous membranes (e.g. nose, eyes, mouth and/or udder), increasing the smoothness and/or shine of the animal's coat, maintaining a regular rate of breathing and/or pulse for that animal, and/or maintaining a regular level of chewing (or ruminating) and/or sweating for that animal.

### Compositions

A compound of Formula (I) may be chemically synthesised or may be isolated from a natural source, for example a plant. The synthesis of glycoglycerol lipids and the like is well known and the skilled person could readily make the compounds of the invention by using and modifying known reaction mechanisms, such as that described in Manzo, E.; Letizia Ciavatta, M.; Pagano, D.; Fontana, A. Tetrahedron Lett. 2012, 53, 879. Alternatively, some glycoglycerol lipids and the like are naturally occurring and so may be isolated from plant materials, for example tomatoes.

Compositions of the present invention may include an extract of a natural material, such as a plant or a fruit of a plant, for example the leaves of plants, or from the pulp of the fruit of plants. For example, Oleaceae, e.g. Olea, such as Olea europaea (olives, in particular from the pulp of the fruit of this plant) Solanaceae, for example Nicotiana (tobacco plants) or Solanum, such as Solanum lycopersicum (the tomato plant), or Amaranthaceae, for example Spinacia, such as Spinacia oleracea (spinach) or Sapotaceae, for example Pouteria, such as Pouteria lucuma (especially from the pulp of the fruit of this plant), or Brassicaceae, such as Arabidopsis, for example Arabidopsis thaliana (especially from the leaves of this plant), for example following bacterial infection or wounding of such samples. The composition may include more than one compound according to Formula (I).

Preferably the compositions include the compound according to Formula (I) in an amount of 50 wt % or more, such as 60 wt % or more, or 70 wt % or more. The compositions may include the compound of Formula (I) in an amount of 80 wt % or more, such as 90 wt % or more. The amount of the compound according to Formula (I) in the composition may be from 50 to 99 wt %, such as from 60 to 98 wt %, or from 60 to 95 wt %, for example from 60 to 90 wt %.

The composition may comprise an eIF4A inhibitor selected from the list consisting of: elatol, hippuristanol, zotatifin, pateamine A, CR-1-31-B and silvestrol.

It will be understood that DGMG is a galactolipid. The composition may comprise other galactolipids, for example a compound according to Formula (II):

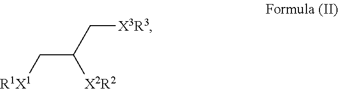

including tautomeric or stereochemically isomeric forms thereof, wherein:
R¹ is selected from a carbohydrate group or a derivative thereof, a C1-C30 alkyl group or a derivative thereof, a C2-C30 alkenyl group or a derivative thereof, and a C2-C30 alkynyl group or a derivative thereof;
R² and R³ each independently represent a hydrocarbon group is selected from a C1-C30 alkyl group or a derivative thereof, a C2-C30 alkenyl group or a derivative thereof, and a C2-C30 alkynyl group or a derivative thereof;
X¹ is a linking group;
X² is a linking group;
X³ is a linking group;
each Y group independently represents a group selected from the list consisting of: cyano, halogen, N₃, —C(O)R^{Z}, —C(O)OR^{Z}, —OC(O)R^{Z}, —C(O)NHR^{Z}, —NHC(O)R^{Z}, —NHC(O)NHR^{Z}, —NHC(O)OR^{Z}, —OC(O)NHR^{Z}, —OP(O)₂OR^{Z}, —S(O)₂NHR^{Z}, —NHS(O)₂R^{Z}, —NR^{Z}₂, —NHR^{Z} and —OR^{Z};
each R^{Z} independently represents C1-10 alkyl, C2-10 alkenyl, C6-10 aryl, or C4-10 heterocyclyl;
or an N-oxide thereof or a pharmaceutically acceptable salt thereof or a pharmaceutically acceptable solvate thereof.

The key difference between Formula (I) and Formula (II) is that Formula (II) includes two hydrocarbon groups whereas Formula (I) only includes one. Otherwise, Formula (II) may include groups as defined for Formula (I).

Preferably, for Formula (II):
i. R¹ represents a monosaccharide group or a disaccharide group; and
ii. R² and R³ each independently represent C6-C30 alkyl or C6-C30 alkenyl, each optionally including one or more Y group;
iii. each Y group independently represents a group selected from the list consisting of: cyano, halogen, N₃, —C(O)R^{Z}, —C(O)OR^{Z}, —OC(O)R^{Z}, —C(O)NHR^{Z}, —NHC(O)R^{Z}, —NHC(O)NHR^{Z}, —NHC(O)OR^{Z}, —OC(O)NHR^{Z}, —OP(O)₂OR^{Z}, —S(O)₂NHR^{Z}, —NHS(O)₂R^{Z}, —NR^{Z}₂, —NHR^{Z} and —OR^{Z}; and
iv. each R^{Z} group independently represents C1-10 alkyl, C2-10 alkenyl, C6-10 aryl, or C4-10 heterocyclyl;
v. each X group independently represents a linker group selected from the list consisting of: ether, thioether, amine, phosphine, ester, thioester, amide, urea, thiourea, carbamate, phosphate and sulfonamide.

The composition may include one or more compound according to Formula (II).

The composition may include a compound according to Formula (II) wherein R¹ represents a monosaccharide in an amount of 1 wt % or more, such as 5 wt % or more, or 10 wt % or more. The composition may include a compound according to Formula (II) wherein R¹ represents a monosaccharide in an amount of 60 wt % or less, such as 50 wt % or less, or 40 wt % or less, preferably 30 wt % or less, or 20 wt % or less. For example, the composition may include a compound according to Formula (II) wherein R¹ represents a monosaccharide in an amount of from 1 to 60 wt %, or from 1 to 40 wt %, such as from 1 to 20 wt %, or from 10 to 20 wt %. Compounds according to Formula (II) wherein R¹ represents a monosaccharide may be termed MGDG.

The composition may include a compound according to Formula (II) wherein R¹ represents a disaccharide in an amount of 1 wt % or more, such as 5 wt % or more, or 10 wt % or more, or 20 wt % or more. The composition may include a compound according to Formula (II) wherein R¹ represents a disaccharide in an amount of 60 wt % or less, such as 50 wt % or less, preferably 40 wt % or less, or 30 wt % or less. For example, the composition may include a compound according to Formula (II) wherein R¹ represents a disaccharide in an amount of from 1 to 60 wt %, or from 1 to 40 wt %, such as from 1 to 30 wt %, or from 10 to 30 wt %. Compounds according to Formula (II) wherein R¹ represents a disaccharide may be termed DGDG.

The composition may include a compound according to Formula (I) and one or more compound according to Formula (II) and in a ratio of 1000:1 to 1:5, such as from 1000:1 to 1:2, preferably from 1000:1 to 1:1, such as from 1000:1 to 2:1, or from 1000:1 to 4:1 respectively and by weight. The ratio may be from 500:1 to 1:5, such as from 200:1 to 1:5, or from 100:1 to 1:5, such as from 50:1 to 1:5, or from 20:1 to 1:5 respectively and by weight. The ratio may be from 50:1 to 1:1, or from 50:1 to 2:1 respectively and by weight.

The composition may include one or more, or all, compounds of Formula (II) selected from the list of: MGDG (C16:0/C18:3), MGDG (C18:3/C18:3), DGDG (C16:0/C18:3), DGDG (C18:3/C18:3), and DGDG (18:3/C20:3). It will be understood that the denomination “C[X]:[Y]” indicates that a hydrocarbon chain (R² and R³) includes [X] carbon atoms and [Y] alkene bonds.

It will be appreciated that, as well as being extracted from natural sources, the present compositions may be formulated by combining samples of the components in the desired amounts.

Unless incompatible, definitions relating to compounds of Formula (I) may used for compounds as defined for Formula (II).

The compounds of the present invention may contain one or more asymmetric carbon atoms (chiral centres) and can therefore exist in racemic and optically active forms. The present invention encompasses all stereoisomeric forms of the compounds. Thus, optical isomers or enantiomers, racemates, diastereomers, and mixtures of diastereomers, are also encompassed in the compounds. The present invention therefore relates to a compound, which may be in the form of an enantiomer, a diastereomer, a racemate, or a mixture of diastereomers, and which may be provided in the form of a pharmaceutically acceptable salt or solvate of the stated Formula. In one embodiment, the product is provided in the form of a mixture of diastereomers; this mixture may have improved solubility properties which in turn can make the compound easier to work with and easier to formulate as a pharmaceutical or nutraceutical composition.

### CLAUSES

The present disclosure includes the subject-matter of the following clauses:

1. A compound of Formula (I) for use as a medicament, wherein Formula (I) is:

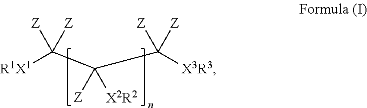

including tautomeric or stereochemically isomeric forms thereof, wherein: R¹ is selected from a carbohydrate group or a derivative thereof, a C1-C30 alkyl group or a derivative thereof, a C2-C30 alkenyl group or a derivative thereof, and a C2-C30 alkynyl group or a derivative thereof; a hydrocarbon group is selected from a C1-C30 alkyl group or a derivative thereof, a C2-C30 alkenyl group or a derivative thereof, and a C2-C30 alkynyl group or a derivative thereof; n is 0 or 1; where n is 0, R³ represents the hydrocarbon group; where n is 1, either: a) R² represents H and R³ represents the hydrocarbon group, or b) R² represents the hydrocarbon group and R³ represents H; X¹ is a linking group; X² is a linking group; X³ is a linking group; each Z group independently represents Y, R^{Z} or H; each Y group independently represents a group selected from the list consisting of: cyano, halogen, N₃, —C(O)R^{Z}, —C(O)OR^{Z}, —OC(O)R^{Z}, —C(O)NHR^{Z}, —NHC(O)R^{Z}, —NHC(O)NHR^{Z}, —NHC(O)OR^{Z}, —OC(O)NHR^{Z}, —OP(O)₂OR^{Z}, —S(O)₂NHR^{Z}, —NHS(O)₂R^{Z}, —NR^{Z}₂, —NHR^{Z} and —OR^{Z}; each R^{Z} independently represents C1-10 alkyl, C2-10 alkenyl, C6-10 aryl, or C4-10 heterocyclyl; or an N-oxide thereof or a pharmaceutically acceptable salt thereof or a pharmaceutically acceptable solvate thereof.

2. The compound of clause 1, wherein Formula (I) represents Formula (Ic):

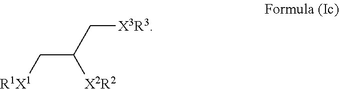

3. The compound of clause 1 or clause 2, wherein R¹ represents a carbohydrate group, preferably a monosaccharide or disaccharide group.

4. The compound of any one of clauses 1 to 3, wherein the hydrocarbon group represents C10-C26 alkyl or C10-C26 alkenyl.

5. The compound of any preceding clause, wherein the hydrocarbon group is alkenyl.

6. The compound of clause 5, wherein the alkenyl hydrocarbon group includes from 1 to 4 alkene groups.

7. The compound of any preceding clause, wherein X¹ represents a linker group selected from the list consisting of: ether, thioether and amide.

8. The compound of clause 7, wherein X¹ represents an ether group.

9. The compound of any preceding clause, wherein the X² or X³ group bonded to the hydrocarbon group represents a linker group selected from the list consisting of: ester, thioester and amide.

10. The compound of clause 9, wherein the X² or X³ group bonded to the hydrocarbon group represents an ester group.

11. The compound of any preceding clause, wherein the X² or X³ group bonded to H is —O— or —S—.

12. The compound of clause 11, wherein the compound of formula (I) is represented by the formula:

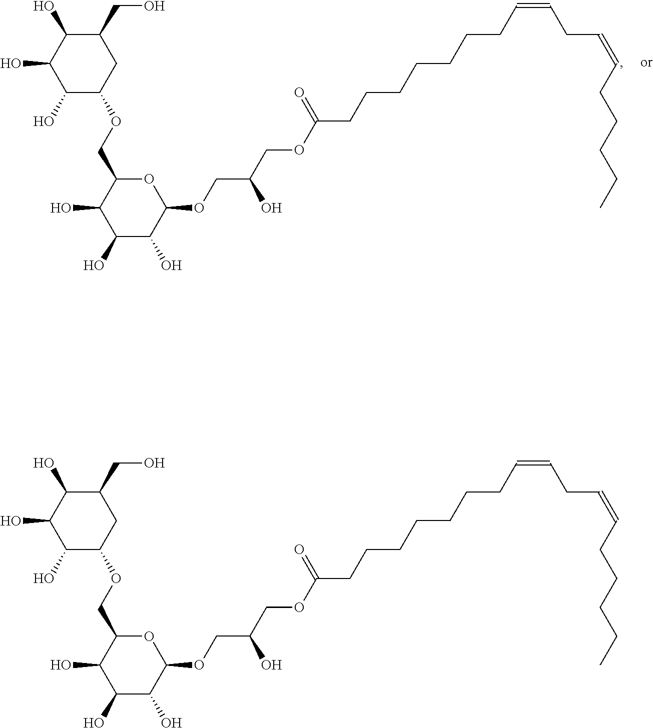 [SMILES: CCCCC/C=C\C/C=C\CCCCCCCC(=O)OC[C@@H](O)CO[C@@H]1O[C@H](CO[C@H]2C[C@H](CO)[C@H](O)[C@H](O)[C@H]2O)[C@H](O)[C@H](O)[C@H]1O ; CCCCC/C=C\C/C=C\CCCCCCCC(=O)OC[C@@H](O)CO[C@@H]1O[C@H](CO[C@H]2C[C@H](CO)[C@H](O)[C@H](O)[C@H]2O)[C@H](O)[C@H](O)[C@H]1O]

13. A compound of formula (I), wherein the compound is for use: in the treatment of a disease or condition which is caused by dysregulation of protein translation, and/or as an inhibitor of protein translation, a chemotherapeutic agent, a cell sensitising agent, an antiproliferative agent, an antiviral agent or an adjuvant, and/or in the treatment of disease or condition selected from the group consisting of: cancer, viral infection, Alzheimer's disease, Parkinson's disease, Huntingdon's disease, muscle wasting and autistic spectrum disorders.

14. The compound of clause 13, wherein the compound is used in a method of treatment comprising administering an eIF4A inhibitor selected from the list consisting of: elatol, hippuristanol, zotatifin, pateamine A, CR-1-31-B and silvestrol.

15. Use of a compound of Formula (I), or a composition thereof, to increase the yield of an animal or herd of animals, or to improve the appearance of the animal or herd thereof.

16. A nutraceutical composition comprising a compound of formula (I), or a pharmaceutically acceptable salt thereof or a pharmaceutically acceptable solvate thereof.

17. A pharmaceutical composition comprising: a compound of formula (I), or a pharmaceutically acceptable salt thereof, or a pharmaceutically acceptable solvate thereof; and a pharmaceutically acceptable carrier, diluent or excipient.

18. A composition for use as a medicament, wherein the composition comprises a compound of Formula (I).

19. A composition comprising a compound of Formula (I) for use: in the treatment of a disease or condition which is caused by dysregulation of protein translation; and/or as an inhibitor of protein translation, a chemotherapeutic agent, a cell sensitising agent, an antiproliferative agent, an antiviral agent or an adjuvant; and/or in the treatment of disease or condition selected from the group consisting of: cancer, viral infection, Alzheimer's disease, Parkinson's disease, Huntingdon's disease, muscle wasting and autistic spectrum disorders.

20. The composition of any one of clauses 16 to 19, wherein the composition includes the compound according to Formula (o in an amount of 50 wt % or more.

21. The composition of any one of clauses 16-20, wherein the composition includes one or more compounds according to Formula (II):

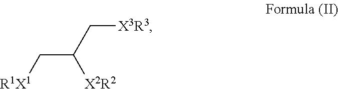

including tautomeric or stereochemically isomeric forms thereof, wherein: R¹ represents a monosaccharide group or a disaccharide group; and R² and R³ each independently represent C6-C30 alkyl or C6-C30 alkenyl, each optionally including one or more Y group; each Y group independently represents a group selected from the list consisting of: cyano, halogen, N₃, —C(O)R^{Z}, —C(O)OR^{Z}, —OC(O)R^{Z}, —C(O)NHR^{Z}, —NHC(O)R^{Z}, —NHC(O)NHR^{Z}, —NHC(O)OR^{Z}, —OC(O)NHR^{Z}, —OP(O)₂OR^{Z}, —S(O)₂NHR^{Z}, —NHS(O)₂R^{Z}, —NR^{Z}₂, —NHR^{Z} and —OR^{Z}; and each R^{Z} group independently represents C1-10 alkyl, C2-10 alkenyl, C6-10 aryl, or C4-10 heterocyclyl; each X group independently represents a linker group selected from the list consisting of: ether, thioether, amine, phosphine, ester, thioester, amide, urea, thiourea, carbamate, phosphate and sulfonamide; or an N-oxide thereof or a pharmaceutically acceptable salt thereof or a pharmaceutically acceptable solvate thereof.

22. The composition of clause 21, wherein the composition includes a compound according to Formula (II) wherein R¹ represents a monosaccharide, in an amount of from 1 wt % to 20 wt %.

23. The composition of clause 21 or clause 22, wherein the composition includes a compound according to Formula (II) wherein R¹ represents a disaccharide, in an amount of from 1 wt % to 30 wt %.

24. A compound for use in a method of treatment, wherein the compound is an eIF4A inhibitor selected from the list consisting of: an anti-cancer agent or an eIF4A inhibitor, wherein the anticancer agent or eIF4A inhibitor is not of Formula (I), and wherein the method comprises administering the eIF4A inhibitor or anti-cancer agent and administering a compound of Formula (I).

25. A kit comprising: a first therapeutic agent comprising a compound of Formula (I), or a composition thereof, and a second therapeutic agent comprising an anti-cancer agent, wherein the anti-cancer agent is provided in a form suitable for, and/or with instructions for, administration in a daily dosage which is significantly reduced (e.g. by 10% or more, or 20% or more, or 30% or more) compared to the dosage of the anti-cancer agent if administered alone. The first and second therapeutic agents may be intended to be administered simultaneously, sequentially or separately. The anti-cancer agent may be a chemotherapeutic agent.

### Examples

MGDG is monogalactosyldiacylglycerol, MGMG is monogalactosylmonoacylglycerol, DGDG is digalactosyldiacylglycerol, and DGMG is digalactosylmonoacylglycerol.

### Extraction of DGMG

FIG. 1a of the accompanying drawings shows the structure of DGMG C18:2, which is a preferred compound of Formula (I). It will be understood that the denomination “C18:2” indicates that the hydrocarbon chain includes 18 carbon atoms and 2 alkene bonds.

FIG. 1b of the accompanying drawings shows the structure of DGMG C16:2, which is another preferred compound of Formula (I).

DGMG may be extracted from plant materials, for example tomatoes. Tomatoes were grown under standard glass house, harvested and frozen Sample preparation: 10 g of tomato pulp was dissolved in 100 mL of chloroform/methanol (2:1, by volume). The resulting solution was agitated for 10 minutes. 20 mL of 0.05M sodium chloride was added and agitated for 10 minutes and left for 1 hour for the phases to fully separate. The aqueous layer was removed, and the organic phase had its solvent removed under reduced pressure to yield a 42.9 mg of extracted tomato lipids. The lipids were taken up in 10 mL of chloroform to give a sample solution of approximately 4000 ppm of extracted tomato lipids.

HPLC: The tomato extracts were submitted to HPLC-CAD analysis. A gradient consisting of an acidic mobile phase of acetonitrile and water+0.1% formic acid was used, as shown below

| Time | Flow | % water + | % |
|---|---|---|---|
| (minutes) | (mL/min) | 0.1% formic acid | Acetonitrile |
| 0 | 1 | 25 | 75 |
| 3 | 1 | 25 | 75 |
| 13 | 1 | 0 | 100 |
| 18 | 1 | 0 | 100 |
| 18 | 1 | 25 | 75 |
| 20 | 1 | 25 | 75 |

Four samples were analysed and were found to contain the following amounts of galactolipids:

Amount of composition in
extraction sample (μg/mL)

| Lipid | 1 | 2 | 3 | 4 |
|---|---|---|---|---|
| DGMG (C18:2) | 1.68 | 2.99 | 1.22 | 2.52 |
| MGDG (C16:0/C18:3) | 0.01 | 0.01 | 0.01 | 0.01 |
| MGDG (C18:3/C18:3) | 0.45 | 0.35 | 0.32 | 0.46 |
| DGDG (C16:0/C18:3) | 0.01 | 0.01 | 0.00 | 0.01 |
| DGDG (C18:3/C18:3) | 0.70 | 0.43 | 0.47 | 0.59 |
| DGDG (18:3/C20:3) | 0.04 | 0.04 | 0.04 | 0.03 |
| [Total MGDG] | 0.46 | 0.35 | 0.33 | 0.48 |
| [Total DGDG] | 0.74 | 0.47 | 0.51 | 0.63 |
| [Total Galactolipids] | 2.88 | 3.82 | 2.07 | 3.62 |

Sample 4, which includes 70% DGMG of total galactolipids, was used in the following tests and is also named AN69T.

Binding of DGMG to eIF4A

The protein-ligand binding free energy value for DGMG, hippuristanol and synthetic derivative zotatifin with eIF4A was determined.

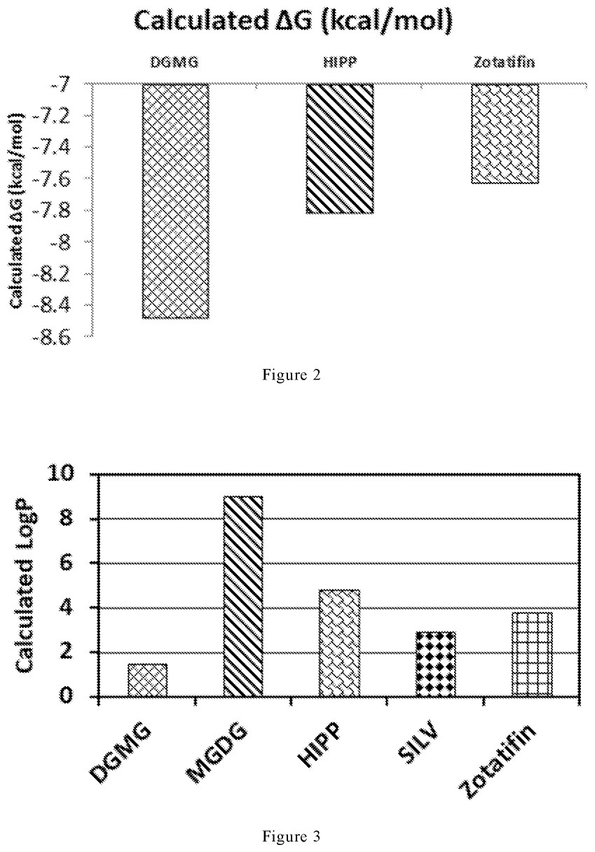

FIG. 2 of the accompanying drawings shows the calculated AG (kcal/mol) protein-ligand binding free energy value for DGMG (16:2), hippuristanol and synthetic derivative zotatifin. The calculated AG (kcal/mol) protein-ligand binding free energy value of DGMG was found to be lower than values calculated for known eIF4A inhibitors hippuristanol and the silvestrol derivative zotatifin. This shows that DGMG bound more strongly than either hippuristanol or zotatifin.

The binding site for DGMG/eIF4A is equivalent to the site reported for hippuristanol (as reported by Lloyd et al, Proteomics. 2021 24; e2000288).

Thus, DGMG has an improved activity compared to other known inhibitors of eIF4A.

cLogP of DGMG

The cLogP values of the eIF4A inhibitors DGMG, MGDG, hippuristanol (HIPP), silvestrol (SILV) and zotatifin were determined.

FIG. 3 of the accompanying drawings shows the cLogP of these eIF4A inhibitors.

It was found that DGMG(16:2) has a cLogP of between 1 and 2, which is lower than any of the other eIF4A inhibitors tested. The cLogP value of DGMG is a more appropriate for pharmaceutical use than any of the other eIF4A inhibitors tested.

### Predicted Off-Target Effects

Predictive in-silico analysis was used to identify the “off-target” profile of DGMG(16:2) and hippuristanol as an ion channel modulator, nuclear receptor ligand and enzyme inhibitor.

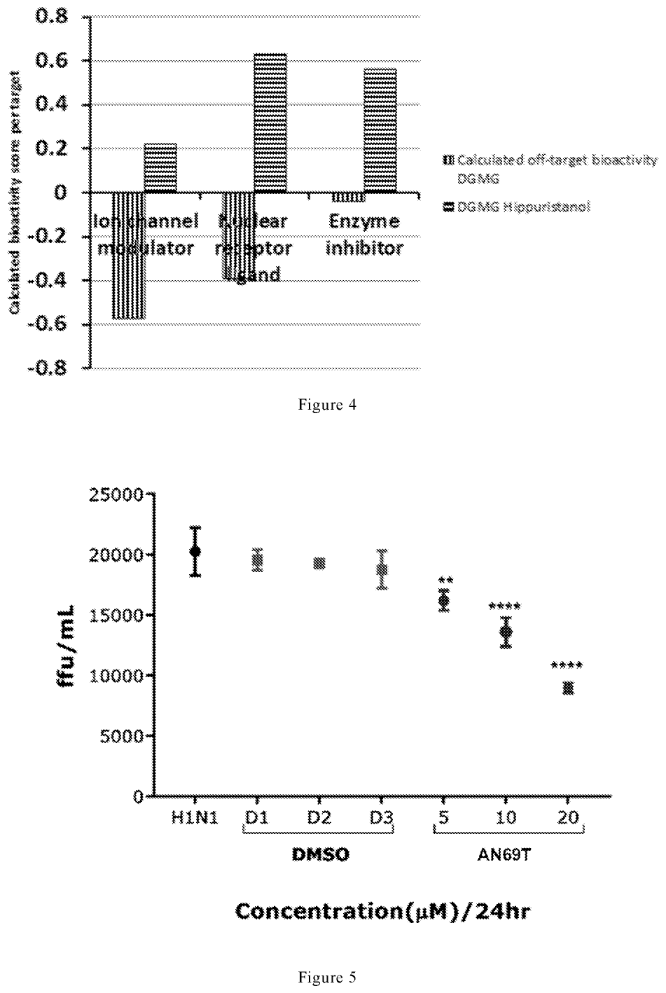

FIG. 4 of the accompanying drawings shows the results of this analysis.

It can be seen that DGMG has a lower off-target bioactivity than hippuristanol as an ion channel modulator, nuclear receptor ligand and enzyme inhibitor.

Based on the above-described cLogP and off-target effects it can be seen that DGMG has improved med-chem properties compared with other inhibitors of eIF4A.

### Treatment of H1N1

AN69T, including DGMG (18:2), was used to treat A549 cells infected with H1N1 (a subtype of Influenza A virus, causing swine flu).

Confluent cells were treated with compound at different concentrations for 1 hr. Cells were then washed with pre-warmed PBS and incubated with virus e.g. H1N1 at the MOI of 0.1 with and without treatment for 24h or 48h post infection (pi) at 37° C. Infectivity determined by a quantitative plaque assay; immune-cytochemical staining for viral nucleoprotein. Statistical significance determined by one-way ANOVA, error bars represent standard deviation.

FIG. 5 of the accompanying drawings shows the results of this study after 24 hours.

After 24 hours post-infection it was found that there was no significant reduction in the vehicle control (DMSO) samples, compared to the untreated samples.

However, in the presence of AN69T, a reduction of viral load was observed at all concentrations tested. The reduction of viral load was greater when the cells were treated with a higher concentration of AN69T, showing that the antiviral effects of AN69T are dependent upon the dose of AN69T.

From these data it can be estimated that the EC₅₀ of AN69T at 24 hours is around 18 μM.

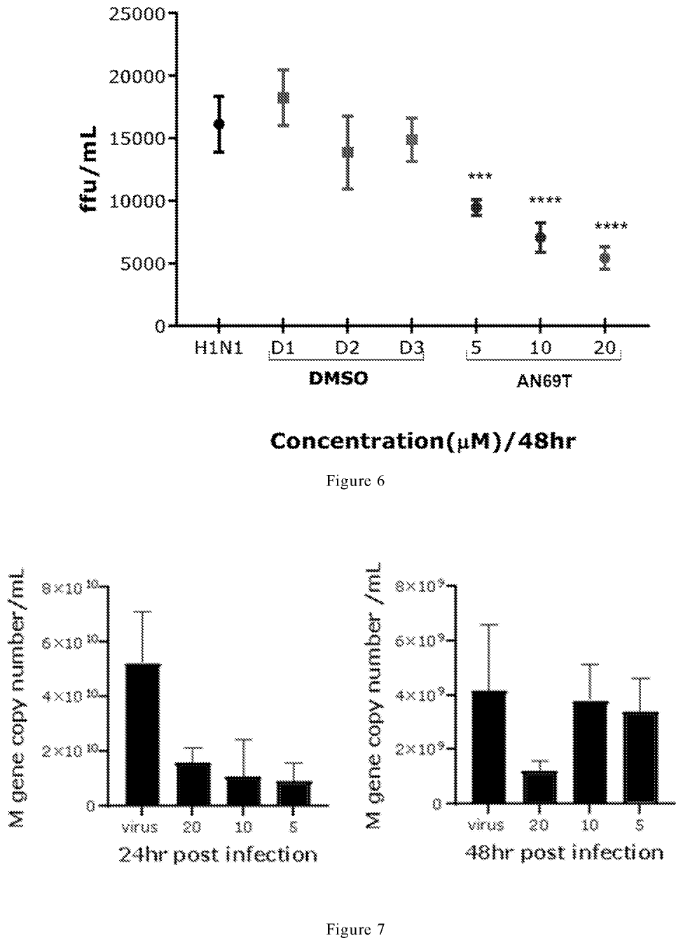

FIG. 6 of the accompanying drawings shows the results of this study after 48 hours.

After 48 hours post-infection it was found that there was no significant reduction in the vehicle control (DMSO) samples, compared to the untreated samples.

However, in the presence of AN69T, a reduction of viral load was observed at all concentrations tested.

The reduction of viral load was greater when the cells were treated with a higher concentration of AN69T, showing that the antiviral effects of AN69T are dependent upon the dose.

From these data it can be estimated that the EC₅₀ of AN69T at 48 hours is around 9 μM.

These data show that there is also a time-dependency to the antiviral activity exhibited by AN69T, since there is a greater inhibition at 48 hours post-infection than at 24 hours post-infection.

The time-dependency of the antiviral activity shows that there is prolonged and sustained activity after 24 hours and to at least 48 hours post-infection. Viruses can alter their mechanism of action to work around antiviral therapies, but the results of the present examples do not exhibit any such alteration to the mechanism of action; the therapy remains effective up to at least 48 hours post-infection.

The dose dependency of the antiviral activity shows that this effect is real and caused by AN69T, which is a composition containing the compound DGMG.

The time-dependency and dose-dependency of the antiviral activity on AN69T fit with the mechanism of action being the inhibition of eIF4A.

### Treatment of LPAI H2N3

Viral load post-infection was quantified using RT-PCR. M gene copy number for Influenza A viruses pre-treated with AN69T at three different concentrations (5, 10 and 20 μM) for 24 and 48 hr post infection. LPAI H2N3 infected MDCK cells at MOI of 0.1. AN69T demonstrated a significant impact on the number of copies of the M gene. Data points show the mean and standard error of triplicate well (*p<0.05; one-way ANOVA).

The results are shown by FIG. 7 of the accompanying drawings.

Treatment with compound results in reduced viral load (RNA levels determined by RT-PCR). Treatment with AN69T significantly inhibited viral replication (specifically the synthesis of M gene RNA). The results show activity after 24 hours as low as 5 μM. The right-hand graph shows that a sustained response at 48 hrs was obtained at 20 μM.

### Treatment of H1N1 and H2N3

Western blot analysis was performed on influenza virus at MOI of 0.1, at 24and 48 hr post-infection. Cell lysates were harvested after 10 mins and probed with Primary and anti-mouse secondary antibody against NS1 viral protein.

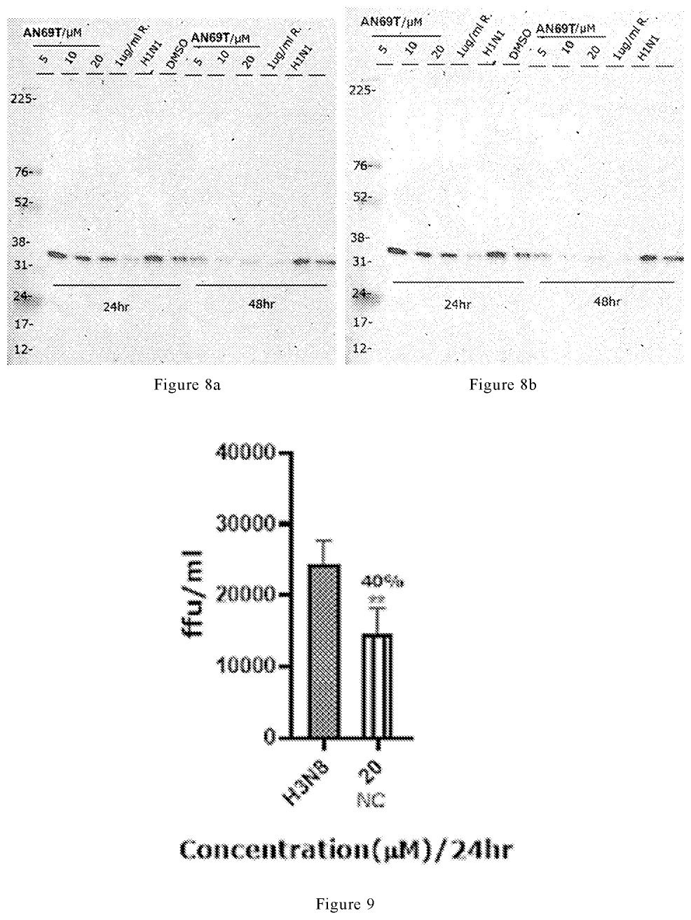

FIG. 8 of the accompanying drawings shows the results of these tests. FIG. 8a shows the results for H1N1. FIG. 8b shows the results of H2N3.

These results show that, for both viruses there was a significant reduction in the viral load following incubation with the compound of the invention after 24 hours, and especially after 48 hours. It again appeared that the higher the dose was the lower the resulting viral load was.

Therefore, compounds of the invention can be used to treat Influenza H1N1 and H2N3.

### Treatment of H3N8

Equine influenza H3N8 is a highly contagious disease that is very common in horses.

Confluent MDCK cells were treated with NC (i.e. AN69T, 20 μM concentration) for 1 hr followed by incubation with H3N8 at a MOI of 0.1, for 24h post-infection at 37° C. Control cells were not treated with NC. The supernatants were each collected at the 24 hours post-infection and virus titre was measured using standard plaque assays in MDCK cells.

The results are shown by FIG. 9 of the accompanying drawings.

The results show a 40% reduction in virus titre after treatment with NC (i.e. reduced infectivity/viral load). Therefore, treatment with NC would likely reduce the symptoms of equine influenza and the excretion (shedding) of the virus after infection.

This data also supports the benefits possible by using NC and related compounds as a feed additive for animals such as horses due to its potential to act as a preventative treatment for viral infections.

### Treatment of Schmallenberg Orthobunyavirus

Schmallenberg virus (SBV-negative single-stranded RNA) is an emerging orthobunyavirus of ruminants associated with outbreaks of congenital malformations in aborted and stillborn animals (Varela et all, 2013). A comparison of plaques produced by SBV treated and untreated with the natural compound was performed.

Vero-E6 cells were infected at a MOI of 0.1 for 24 hrs. Supernatants were collected at the 24 hours post-infection and virus titre was measured using standard plaque assays in vero-E6 cells.

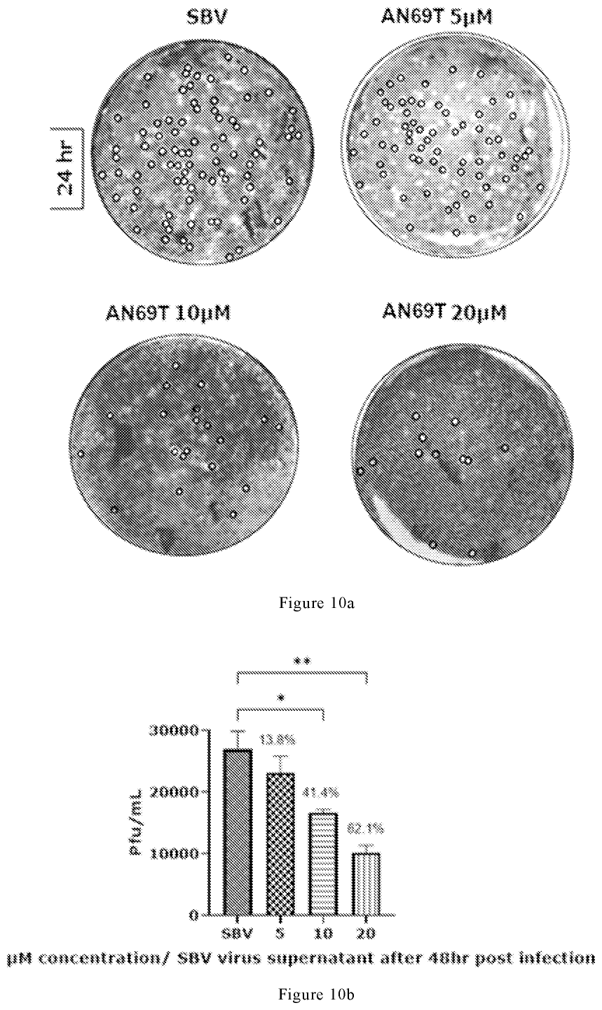

The results are shown by FIG. 10 of the accompanying drawings. FIG. 10a specifically shows images of the plates with overlaid analysis showing the plaques observed. FIG. 10b shows a graph indicating the amount of virus detected in each plate.

It can be seen that there is a reduction in the concentration of viral plaques in all tests according to the invention, compared to the control. A slight (13.8%) reduction in the concentration of viral plaques was observed for the 5 μM plate, whereas more significant reductions (41.4% and 62.1%) reductions were observed for the 10 μM and 20 μM plates respectively.

Therefore, compounds of the invention can be used to treat Schmallenberg orthobunyavirus.

### Treatment of Zika Virus (ZIKV)

Plaques produced by Vero-E6 cells infected at a MOI of 0.1 by ZIKV treated (at 5 μM, 10 μM or 20 μM) or untreated with AN69T were compared after 24 or 48 hrs. Supernatants were collected at the indicated times post-infection and virus titre was measured using standard plaque assays in vero-E6 cells.

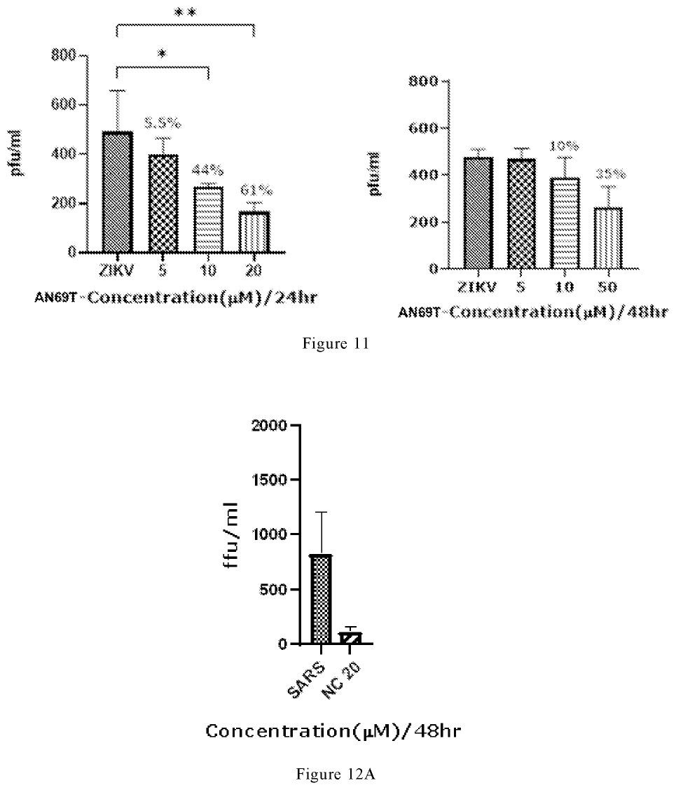

The results are shown in FIG. 11 of the accompanying drawings. The left-hand graph shows that, after 24 hours, there was a reduction in the viral load for all samples of the invention. The reduction increased according to the concentration of AN69T, providing a 61% reduction at 20 μM. There was less reduction after 48 hours (right-hand graph), but the 10 and 50 μM samples still exhibited some reduction in viral load.

Thus, compounds of the invention can be used to treat Zika Virus.

### Treatment of SARS-CoV-2

Confluent MDCK cells were treated with NC (i.e. AN69T, 20 μM concentration) for 1 hr followed by incubation with SARS-CoV-2 (i.e. COVID-19) at the MOI of 0.1 for 24 hrs and 48h post infection (pi) at 37° C. Control cells were not treated with NC. Supernatants were collected and assessed via a focus forming assay.

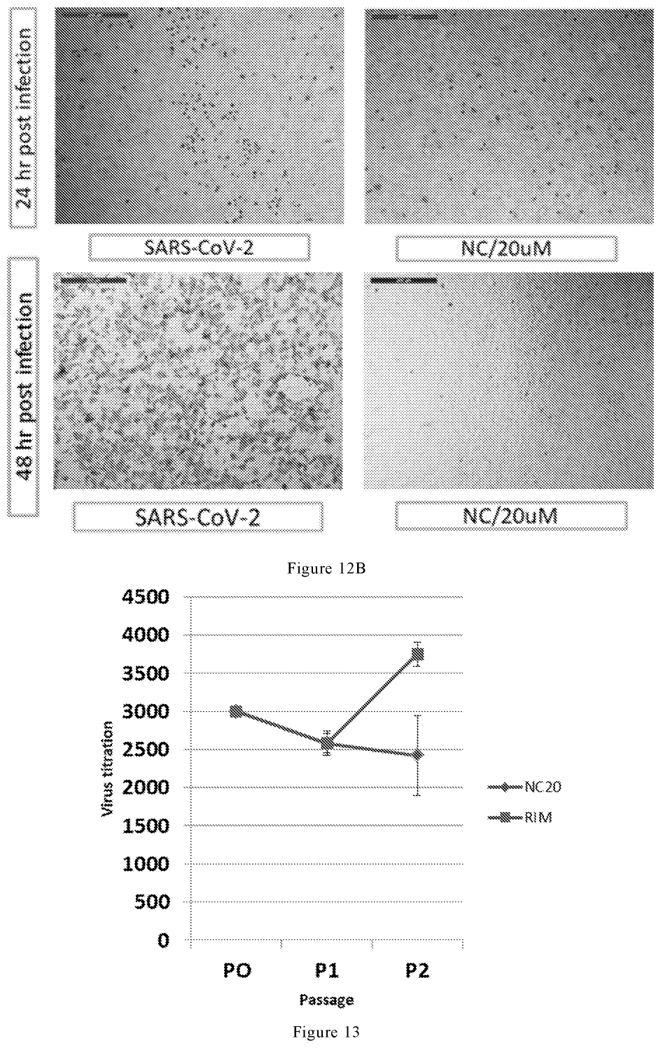

FIG. 12A of the accompanying drawings shows the amount of viral focus-forming units (FFU) 48 hours after treatment with NC. The vial titre was significantly reduced in the cell sample treated with NC, compared to the control, showing that NC can be used to treat SARS-CoV-2 infections.

FIG. 12B of the accompanying drawings shows representative images of immuno-stained cell assay/viral infection at 24 h and 48 h post-infection, with and without treatment by NC. Dots on the images show stained infected cells. There is a significant reduction in the amount of infected cells in the treated samples. Compared to the control sample (untreated), the treated sample shows an even greater difference in the amount of infected cells after 48 hours than after 24 hours. Over this period, the number of infected cells increases in the untreated control sample, whereas the number of infected cells decreases in the treated sample. This suggests prolonged efficacy of the treatment.

Overall, these results show that treatment with NC can reduce viral load and block infectivity of SARS-CoV-2.

### Treatment of H1N1 at Low Dose Over Serial Passage

Serial passage of a virus allows the virus to rapidly evolve to its host, and is used to study the evolution of resistance.

Rimantadine is an FDA-approved drug used in-clinic to prevent and treat infections caused by the influenza A virus.

Confluent MDCK cells were treated with either NC (20 μM) or 1 μg/ml rimantadine (RIM) for 1 hr followed by incubation of the cells with H1N1 at the MOI of 0.1 for 48 h pi at 37° C. Following this, the supernatants were collected and virus titre was measured using standard plaque assays in MDCK cells.

FIG. 13 of the accompanying drawings shows the results of the low dose serial passage study.

Viral titre was similar for NC and RIM in passages P0 and P1. Both samples showed a decrease in viral titre from passage P0 to passage P1. However, in passage P2, viral titre increased significantly for the sample treated with RIM. By contrast, the sample treated with NC continued to decrease in viral titre.

This shows that resistance to RIM increased during passage P2, whereas no resistance was observed in response to repeat treatment with NC. It is postulated that the mechanism of action of the compounds of the invention allows them to remain effective despite viral evolution processes.

### Mechanism of Action Study

It is postulated that the compounds of the invention inhibit viral protein synthesis by inhibiting eIF4A, which in turn inhibits the initiation of translation, which involves. The requirement for eIF4A activity during translation for a virus is dependent upon the viral 5′UTR mRNA structure.

Folding free energy represents difference in free energy between the unfolded and folded state. For any given virus, a lower 5′-UTR mRNA folding free energy corresponds to a more stable secondary structure and a greater dependency on eIF4A. The 5′-UTR length and folding free energy of a virus can therefore be used to approximate the dependence of the virus upon eIF4A in its translation. For example, coronaviruses such as SARS-CoV-2, MERS and SARS have hairpin structured 5′-UTRs that are sensitive to eIF4A inhibition. As shown above, the present invention has been shown to be effective against infections of a variety of viruses.

Promisingly, the sequences of SARS-CoV-2 variants all have highly conserved 5′-UTR mRNA structures, meaning that they are equally dependent upon, and sensitive to the inhibition of, eIF4A.

For a range of viruses, the relative efficacy of NC was compared with the approximate 5′-UTR length and the 5′-UTR folding free energy to determine whether there was a correlation between the eIF4A dependency of the virus and the efficacy of NC against the virus. A correlation would indicate that the NC inhibits the action of eIF4A.

The table below shows the 5′-UTR folding free energies of three viruses against the approximate percentage inhibition by AN69T at 20 μM.

| | Virus | | |
|---|---|---|---|
| | Influenza | ZIKA | COVID-19 |
| Approximate 5′-UTR length | 40 | 107 | 265 |
| (base pairs) | | | |
| 5′-UTR folding free energy | −10.4 | −28.1 | −82.1 |
| (kcal/mol) | | | |
| Approximate inhibition at 20 μM | 50 | 61 | 85 |
| (%) | | | |

NB: Influenza utilises a host RNA cap snatching approach to generate 5′-UTRs for transcripts. Influenza sequence reference: RNA. 2015 December; 21(12): 2067-2075. Analysis of the snatching rate (5′UTR) of top abundant host RNA in the 24-h infected sample. Zika sequence reference: GenBank: KY583506.1. Schmallenberg sequence reference: GenBank: JX853179.1. COVID-19 sequence reference: GenBank: MT192772.1.

This shows that the inhibitory effects of NC against a virus correlate with the 5′-UTR structure, and the eIF4A dependency, of the virus.

This confirms that the effects of NC are consistent with in-silico analysis of the target engagement, mechanism of action and target activity, as discussed above. Moreover, longer UTRs tend towards a greater requirement for eIF4A. Inhibition with NC correlates with both 5′-UTR length and free energy in these test viruses.

Thus, this data indicates that NC inhibits the action of eIF4A.

### Body Weight Change

Two cohorts of mice were fed the same feed composition. One cohort was administered, via gavage, NC (5.5 mg per kg per day). The other cohort (control) was administered DMSO via gavage as an equivalent vehicle control. The weight of each subject was measured daily over the course of 5 days.

The body weight of each test subject was compared to the average body weights of control mice. The table below shows the relative percentage difference in body weight for each test mouse, relative to the control group.

Mouse test subject

| Day | ID1 | ID2 | ID3 | ID4 |
|---|---|---|---|---|
| 0 | 0% | 0% | 0% | 0% |
| 4 | 27% | 11% | 19% | 3% |
| 5 | 38% | 19% | 19% | 7% |

It can be seen that each mouse significantly increased in body weight throughout the study, due to being fed with NC rather than the control diet.

There were significant increases in body weight between days 0 and 4. In addition, there were surprisingly significant increases in body weight between days 4 and 5, suggesting that the effects achieved while being fed NC are experienced even several days after the first feeding session.

This shows that compounds of the invention can be used to increase the yield or appearance of an animal or herd of animals.

### CONCLUSIONS

It has been shown that the compounds of the invention act by inhibiting eIF4A. Thus, it can be expected that these compounds can treat or prevent diseases that are treated by inhibiting eIF4A.

One example of a disease that is treated or prevented by the inhibition of eIF4A is viral infections.

These data show that compounds of the claimed invention exhibit broad antiviral activity. This has been illustrated for multiple subtypes of influenza, Schmallenberg virus, Zika virus, and SARS-CoV-19.

As well as being effective against a wide variety of viruses, the compounds of the invention have been demonstrated to be resilient against the evolution of resistance in viruses.

In addition, it has been shown that compounds of the invention can be used to significantly increase the yield or appearance of animals.

1. A method comprising administering to a subject a compound of Formula (I), wherein Formula (I) is: 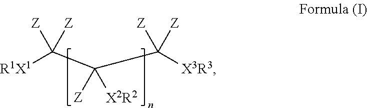
including tautomeric or stereochemically isomeric forms thereof, wherein:
R¹ is selected from a carbohydrate group or a derivative thereof, a C1-C30 alkyl group or a derivative thereof, a C2-C30 alkenyl group or a derivative thereof, and a C2-C30 alkynyl group or a derivative thereof;
a hydrocarbon group is selected from a C1-C30 alkyl group or a derivative thereof, a C2-C30 alkenyl group or a derivative thereof, and a C2-C30 alkynyl group or a derivative thereof;
n is 0 or 1;
where n is 0, R³ represents the hydrocarbon group;
where n is 1, either: a) R² represents H and R³ represents the hydrocarbon group, or b) R² represents the hydrocarbon group and R³ represents H;
X¹ is a linking group;
X² is a linking group;
X³ is a linking group;
each Z group independently represents Y, R^{Z} or H;
each Y group independently represents a group selected from the list consisting of: cyano, halogen, N₃, —C(O)R^{Z}, —C(O)OR^{Z}, —OC(O)R^{Z}, —C(O)NHR^{Z}, NHC(O)R^{Z}, —NHC(O)NHR^{Z}, —NHC(O)OR^{Z}, —OC(O)NHR^{Z}, —OP(O)₂OR^{Z}, ˜S(O)₂NHR^{Z}, —NHS(O)₂R^{Z}, —NR^{Z}, —NHR^{Z} and —OR^{Z};
each R independently represents C1-10 alkyl, C2-10 alkenyl, C6-10 aryl or C4-10 heterocyclyl;
or an N-oxide thereof or a pharmaceutically acceptable salt thereof or a pharmaceutically acceptable solvate thereof.

2. The method of claim 1, wherein Formula (I) represents Formula (Ic): 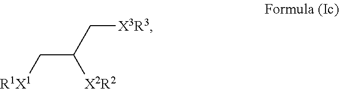
and wherein:
R¹ represents a monosaccharide or disaccharide group;
either:
a) R² represents H and R³ represents the hydrocarbon group, or
b) R² represents the hydrocarbon group and R³ represents H;
the hydrocarbon group represents C10-C24 alkyl or C10-C24 alkenyl;
X¹ and the X group bonded to the hydrocarbon group represents a linker group selected from the list consisting of: ether, ester, amide, urea, and carbamate; and
the X group bonded to H represents a group selected from the list consisting of: —O—, —S—, —C(O)O—, and —C(O)NH—.

3. The method of claim 2, wherein:
R¹ represents a disaccharide group;
R² represents H;
R³ represents the hydrocarbon group;
the hydrocarbon group represents C10-C24 alkyl or C10-C24 alkenyl;
X¹ and X³ represent a linker group selected from the list consisting of: ether, ester, amide, urea, carbamate; and
X² represents a group selected from the list consisting of: —O—, —S—, —C(O)O— and —C(O)NH—.

4. The of claim 1, wherein the hydrocarbon group represents C10-C26 alkyl or C10-C26 alkenyl.

5. (canceled)

6. The method of claim 4, wherein the compound of formula (I) is represented by the formula:

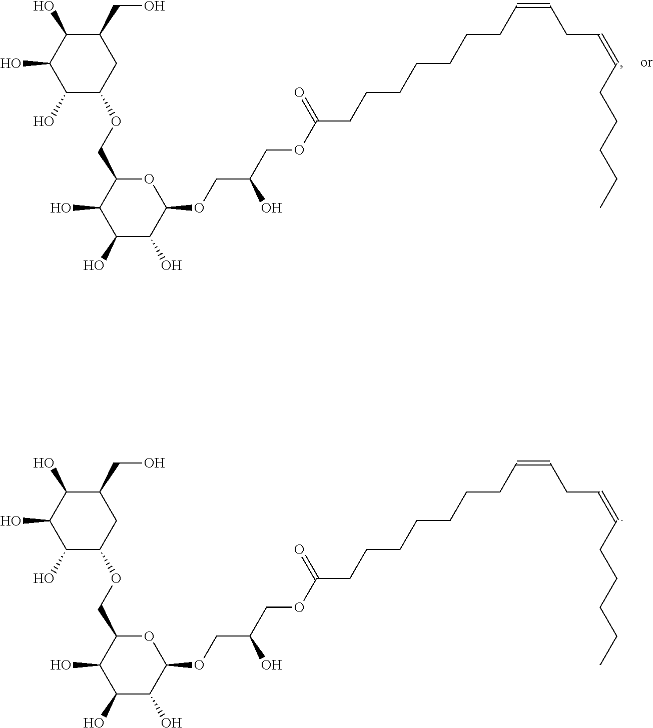 [SMILES: CCCCC/C=C\C/C=C\CCCCCCCC(=O)OC[C@@H](O)CO[C@@H]1O[C@H](CO[C@H]2C[C@H](CO)[C@H](O)[C@H](O)[C@H]2O)[C@H](O)[C@H](O)[C@H]1O ; CCCCC/C=C\C/C=C\CCCCCCCC(=O)OC[C@@H](O)CO[C@@H]1O[C@H](CO[C@H]2C[C@H](CO)[C@H](O)[C@H](O)[C@H]2O)[C@H](O)[C@H](O)[C@H]1O]

7. The method of claim 1, wherein the method is:
a method of treatment or prevention of a disease or condition which is caused by dysregulation of protein translation, and/or
a method of inhibiting protein translation, a method of treating cancer, a method of sensitising cells, a method of inhibiting cellular, parasitic or viral growth, and/or
a method of treatment or prevention of a disease or condition selected from the group consisting of: cancer, viral infection, and a CNS-related disorder.

8. The method of claim 7, wherein the method is inhibiting eIF4A.

9. The method of claim 7, wherein the method is a method of treatment or prevention of Alzheimer's disease, Parkinson's disease, Huntingdon's disease, frontotemporal dementia, motor neurone disease, muscle wasting and autism spectrum disorder.

10. (canceled)

11. The method of claim 7, wherein the method is a method of treatment or prevention of a viral infection, wherein the viral infection is infection with an influenza virus, virus having a genus Flavivirus, a virus having genus orthobunyavirus, or a coronavirus.

12-14. (canceled)

15. The nutraceutical composition of claim 30, wherein the composition is an animal feed.

16. (canceled)

17. The nutraceutical composition of claim 15, wherein the animal feed comprises:
(a) either:
(i) the compound of Formula (I) in an amount of from 0.00001 wt % or more, or
(ii) tomatoes in a dry amount of from 0.001 wt % or more; and
(b) other components selected from the group consisting of: vitamins, minerals, chemical preservatives, antibiotics, fermentation products, molasses, grain, seaweed, fodder, peanut shell, bean pods, corn bract and corn cobs,
wherein the other components are included in an amount of 30 wt % or more.

18-21. (canceled)

22. The composition of claim 28, wherein the composition further comprises at least one compound according to Formula (II): 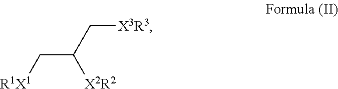
including tautomeric or stereochemically isomeric forms thereof, wherein:
i. R¹ represents a monosaccharide group or a disaccharide group; and
ii. R² and R³ each independently represent C6-C30 alkyl or C6-C30 alkenyl, each optionally including one or more Y group;
iii. each Y group independently represents a group selected from the list consisting of: cyano, halogen, N₃, —C(O)R^{Z}, —C(O)OR^{Z}, —OC(O)R^{Z}, —C(O)NHR^{Z}, —NHC(O)R^{Z}, —NHC(O)NHR^{Z}, ˜NHC(O)OR^{Z}, —OC(O)NHR^{Z}, —OP(O)₂OR^{Z}, —S(O)₂NHR^{Z}, —NHS(O)₂R^{Z}, —NR^{Z}, —NHR^{Z} and —OR^{Z}; and
iv. each R^{Z} group independently represents C1-10 alkyl, C2-10 alkenyl, C6-10 aryl, or C4-10 heterocyclyl;
v. each X group independently represents a linker group selected from the list consisting of: ether, thioether, amine, phosphine, ester, thioester, amide, urea, thiourea, carbamate, phosphate and sulfonamide;
or an N-oxide thereof or a pharmaceutically acceptable salt thereof or a pharmaceutically acceptable solvate thereof.

23. The composition of claim 22, wherein the composition includes:
(a) a compound according to Formula (II) wherein R1 represents a monosaccharide, in an amount of from 1 wt % to 20 wt %; and/or
(b) a compound according to Formula (II) wherein R¹ represents a disaccharide, in an amount of from 1 wt % to 30 wt %.

24-25. (canceled)

26. The method of claim 7, wherein the method further comprises administering an anti-cancer agent or an eIF4A inhibitor, wherein the anticancer agent or eIF4A inhibitor is not a compound of Formula (I).

27. The method of claim 26, wherein the eIF4A inhibitor is selected from the group consisting of elatol, hippuristanol, zotatifin, pateamine A, CR-1-31-B and silvestrol.

28. A composition comprising a compound of Formula (I), wherein Formula (I) is: 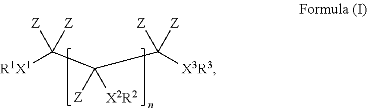
including tautomeric or stereochemically isomeric forms thereof, wherein:
R¹ is selected from a carbohydrate group or a derivative thereof, a C1-C30 alkyl group or a derivative thereof, a C2-C30 alkenyl group or a derivative thereof, and a C2-C30 alkynyl group or a derivative thereof;
a hydrocarbon group is selected from a C1-C30 alkyl group or a derivative thereof, a C2-C30 alkenyl group or a derivative thereof, and a C2-C30 alkynyl group or a derivative thereof;
n is 0 or 1;
where n is 0, R³ represents the hydrocarbon group;
where n is 1, either: a) R² represents H and R represents the hydrocarbon group, or b) R² represents the hydrocarbon group and R3 represents H;
X¹ is a linking group;
X² is a linking group;
X³ is a linking group;
each Z group independently represents Y, R^{Z} or H;
each Y group independently represents a group selected from the list consisting of: cyano, halogen, Ns, —C(O)R^{Z}, —C(O)OR^{Z}, —OC(Q)R^{Z}, —C(O)NHR^{Z}, —NHC(O)R^{Z}, —NHC(O)NHR^{Z}, —NHC(O)OR^{Z}, —OC(O)NHR^{Z}, —OP(O)₂OR^{Z}, —S(O)₂NHR^{Z}, —NHS(O)₂R^{Z}, —NR^{Z}₂, —NHR^{Z} and —OR^{Z};
each R^{Z} independently represents C1-10 alkyl, C2-10 alkenyl, C6-10 aryl, or C4-10 heterocyclyl;
or an N-oxide thereof or a pharmaceutically acceptable salt thereof or a pharmaceutically acceptable solvate thereof.

29. A method of increasing yield of an animal or herd of animals, or to improve the appearance of the animal or herd thereof, comprising administering to the animal or herd of animals the composition of claim 28.

30. A nutraceutical composition, comprising the composition of claim 28.

31. A pharmaceutical composition, comprising the composition of claim 28, and
a pharmaceutically acceptable carrier, diluent or excipient.

32. The composition of claim 28, comprising the compound of Formula (I) in an amount of 50 wt % or more.
